(19) United States
(12) Patent Application Publication
(10) Pub. No.: US 2025/0195427 A1
(43) Pub. Date: Jun. 19, 2025

(54) ANTI-INFLAMMATORY DRUG-ELUTING COMPOSITIONS AND METHODS

(71) Applicant: THE REGENTS OF THE UNIVERSITY OF CALIFORNIA, Oakland, CA (US)

(72) Inventors: Nasim Annabi, Los Angeles, CA (US); Shima Gholizadeh, Los Angeles, CA (US); Xi Chen, San Mateo, CA (US)

(21) Appl. No.: 18/846,446

(22) PCT Filed: Mar. 15, 2023
(86) PCT No.: PCT/US2023/064408

§ 371 Date: Sep. 12, 2024

Related U.S. Application Data
(60) Provisional application No. 63/320,317, filed on Mar. 16, 2022.

(51) Int. Cl. A61K 9/107; A61K 9/00; A61K 31/567; A61K 31/573

(52) U.S. Cl. CPC A61K 9/1075; A61K 9/0048; A61K 31/567; A61K 31/573

(57) ABSTRACT

The present disclosure describes compositions that can be used in the treatment of an ocular injury or disease in a subject in need thereof. Methods of preparing the compositions are also described. The compositions can include one or more micelles encapsulating a hydrophobic therapeutic agent; gelatin methacryloyl (GelMA); hyaluronic acid-glycidyl methacrylate (HAGM); and a visible light-activated photoinitiator.

### FEDERALLY SPONSORED RESEARCH

This invention was made with government support under Grant Number W81XWH-18-1-0654, awarded by the U.S. Department of Defense. The government has certain rights in the invention.

### TECHNICAL FIELD

The present disclosure describes compositions comprising photocrosslinkable gelatin-based composite adhesive hydrogels incorporating micelles. The disclosure also describes methods of treating an ocular injury or ocular disease in a subject in need thereof with these compositions and methods of preparing the compositions. The compositions can include polymeric micelles (MCs), gelatin methacryloyl (GelMA), hyaluronic acid-glycidyl methacrylate (HAGM), and a visible light-activated photoinitiator. The methods can include contacting the eye of the subject with the composition and photo-crosslinking the composition by exposing the composition to a visible light.

### BACKGROUND

Eye diseases have been a serious problem affecting many people around the world due to the changes in lifestyle including but not limited to increase in use of contact lens, longer exposure time to air conditioners, long hours staring at computers, and an increasingly aging population. The above-mentioned conditions can cause dry eyes and irritation and can further develop infections and inflammations like conjunctivitis, glaucoma as well as age-related macular degeneration. Often times, severe eye complications and injuries are treated with eye surgery and post-surgery care is required to prevent inflammations. However, effective and efficient delivery of therapeutics into patient eyes remains a challenge because of several structural barriers such as the corneal epithelium and blood-retinal barrier. Systemic administration routes require a large dose in order to achieve a satisfactory drug concentration at the ocular tissue, which can lead to off-target systemic side effects. On the other hand, local drug delivery such as conventional topical administration (eye drops or ointments) generally have extremely low bioavailability of <5% due to corneal epithelium barrier and fast clearance by tear film and blinking. As a result, repetitive drug applications are required, which may induce ocular hypertension and are also associated with poor patient compliance. Additionally, intraocular injection is used to circumvent the bioavailability issue. For example, surgeons use a small gauge needle to inject the medicine into the patient's eyes after the surgery. However, this method is more invasive and complications such as post-operative pain, intraocular pressure spikes, and retinal detachment can arise. Hence, there is a need for a localized, noninvasive, and efficient ocular drug delivery vehicle and method capable of having a high patient compliance.

### SUMMARY

Certain aspects of the present disclosure are directed to a composition, comprising: one or more micelles encapsulating a hydrophobic therapeutic agent; gelatin methacryloyl (GelMA); hyaluronic acid-glycidyl methacrylate (HAGM); and a visible light-activated photoinitiator.

In some embodiments, the one or more micelles are block copolymer micelles comprising a hydrophobic core and a hydrophilic shell. In some embodiments, the block copolymer is poly(ethylene glycol)-b-poly(N-(2-hydroxypropyl) methacrylamide-oligolactate) (mPEG-b-p(HPMAm-Lacn)). In some embodiments, the hydrophobic therapeutic agent is a first therapeutic agent, and the composition further comprises a second therapeutic agent. In some embodiments, the first and/or second therapeutic agents are anti-inflammatory therapeutic agents. In some embodiments, the first and/or second therapeutic agents are loteprednol etabonate (LE), prednisolone acetate (PA), dexamethasone (DEX), or any combination thereof.

In some embodiments, the one or more micelles have a diameter ranging from about 50 nanometers (nm) to about 150 nm. In some embodiments, the GelMA is present at a concentration ranging from 3% (w/v) to about 14% (w/v). In some embodiments, the HAGM is present at a concentration ranging from about 0.5% (w/v) to about 3% (w/v). In some embodiments, the composition has a sustained therapeutic release profile over a period of about 15 days at most. In some embodiments, the photoinitiator comprises Eosin Y, triethanolamine (TEA), N-vinylcaprolactam (VC), or any combination thereof. In some embodiments, the composition is in a form of a solution or a hydrogel. In some embodiments, the composition further comprises a pharmaceutically acceptable carrier or excipient. In some embodiments, the composition is formulated for topical use. In some embodiments, the one or more micelles are present at a concentration of about 5 mg/ml to about 30 mg/ml.

Certain aspects of the present disclosure are directed to a method of treating an ocular disease or an ocular injury in an eye of a subject, the method comprising: contacting the eye of the subject with any of the composition of the disclosure; and photo-crosslinking the composition by exposing the composition to a visible light.

In some embodiments, the ocular disease is an ocular anterior segment disease or an ocular posterior segment disease. In some embodiments, the ocular disease is conjunctivitis, blepharitis, glaucoma, or a cataract. In some embodiments, the ocular injury is an ocular surface injury or an injury or trauma caused by an ocular surgery. In some embodiments, the visible light has a wavelength of about 400 nanometers (nm) to 800 nm.

Certain aspects of the present disclosure are directed to a method of preparing any of the compositions disclosed herein, the method comprising: dissolving the GelMA and the HAGM in a first solution comprising the visible light-activated photoinitiator; and mixing a second solution comprising the one or more micelles with the dissolved GelMA and HAGM in the first solution.

In some embodiments, the method further comprises mixing a therapeutic agent with the one or more micelles prior to forming the first solution. In some embodiments, the first solution and the second solution further comprise a buffer, a solvent, water, or any combination thereof. In some embodiments, the method further comprises incubating the first solution in the presence of the GelMA and the HAGM for at least about 12 hours prior to mixing with the second solution. In some embodiments, the method further comprises photo-crosslinking the first and second solutions after mixing the second solution with the dissolved GelMA and HAGM in the first solution by exposing the composition to a visible light. In some embodiments, the composition changes from a solution form to a hydrogel form after photo-crosslinking the first and second solutions.

The term “ocular surface injury,” as used herein, can include ulcers, lacerations, defects, perforations, or intentionally performed incisions (e.g., as is done in surgery) of the cornea or sclera.

The terms “subject” or “patient” as used herein refer to any mammal (e.g., a human or a veterinary subject, e.g., a dog, cat, horse, cow, goat, sheep, mouse, rat, or rabbit) to which a composition or method of the present disclosure may be administered, e.g., for experimental, diagnostic, prophylactic, and/or therapeutic purposes. The subject may seek or need treatment, require treatment, is receiving treatment, will receive treatment, or is under care by a trained professional for a particular disease or condition.

The term “composition” as used herein can refer to a precursor composition (e.g., a composition before crosslinking polymerization) and/or a sealant gel composition (e.g., a composition after crosslinking polymerization), as provided by the corresponding context of the disclosure.

As used herein, the term “micelle” includes without limitation micelles having shapes of spheres, cylinders, discs, needles, cones, vesicles, globules, rods, ellipsoids, and any other shape that a micelle can assume under the conditions described herein, or any other shape that can be adopted through aggregation of the amphiphilic copolymers.

As used herein, the singular forms “a,” “an,” and “the” include plural referents unless the context clearly dictates otherwise. Thus, for example, reference to “a micelle” includes mixtures of micelles, reference to “a micelle” includes mixtures of two or more such micelles, and the like.

As used herein, the term “shell” means the outermost domain or peripheral layer of a micelle of the present disclosure. When produced in a hydrophilic continuous medium, the peripheral layer of the micelles is substantially hydrophilic; when produced in a hydrophobic continuous medium, the peripheral layer of the micelles is substantially hydrophobic.

As used herein, the term “core” means the interior domain (e.g., the interior cavity and/or the interior layer) of a micelle, which is interior to the shell.

As used herein, the term “amphiphilic copolymer” means a copolymer which contains at least one hydrophilic domain and at least one hydrophobic domain.

As used herein, the term “block copolymer” means a linear polymer having regions or blocks along its backbone chain that are characterized by similar hydrophilicity, hydrophobicity, or chemistry. The term “diblock copolymer” means a block copolymer comprising two homopolymer subunits linked by covalent bonds. The term “triblock copolymer” means a block copolymer comprising three homopolymer subunits linked by covalent bonds. The term “multiblock copolymer” means a block copolymer comprising a plurality of homopolymer subunits linked by covalent bonds.

As used herein, the term “therapeutic agent” is any molecule or atom which is encapsulated, conjugated, fused, dispersed, embedded, mixed, or otherwise affixed to any of the compositions described herein and is useful for a disease therapy.

As used herein, the expression “pharmaceutically acceptable” applies to a composition which contains composition ingredients that are compatible with other ingredients of the composition as well as physiologically acceptable to the recipient (e.g., a mammal such as a human) without the resulting production of excessive undesirable and unacceptable physiological effects or a deleterious impact on the mammal being administered the pharmaceutical composition. A composition as described herein can comprise one or more carriers, useful excipients, and/or diluents.

As used herein, the term “hydrogel” refers to a broad class of polymeric materials that may be natural or synthetic, have an affinity for an aqueous medium, and are able to absorb large amounts of aqueous medium, but which do not normally dissolve in the aqueous medium.

As used herein, the term “aqueous medium” as used herein refers to water or a solution based primarily on water such as phosphate-buffered saline (PBS), or water containing one or more salts dissolved therein.

As used herein, the term “photo-crosslink” refers to an interconnection between polymer chains via chemical bonding, such as, but not limited to, covalent bonding, ionic bonding, or affinity interactions that are caused by exposure to a light source. The chemical cross-linking can be carried out by reactions, such as any one of free radical polymerization, condensation polymerization, anionic or cationic polymerization, or step growth polymerization.

As used herein, the term “biodegradable” refers to a substance which may be broken down by microorganisms, or which spontaneously breaks down over a relatively short time (within about 14 days to about 6 months) when exposed to environmental conditions commonly found in nature. For example, the compositions described herein may be degraded by enzymes that are present in the body (e.g., the ocular environment).

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. Furthermore, the use of the term “about,” as used herein, refers to an amount that is near the stated amount by about 10%, 5%, or 1%, including increments therein. For example, “about” can mean a range including the particular value and ranging from 10% below that particular value and spanning to 10% above that particular value.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Where values are described in the present disclosure in terms of ranges, endpoints are included. Furthermore, it should be understood that the description includes the disclosure of all possible sub-ranges within such ranges, as well as specific numerical values that fall within such ranges irrespective of whether a specific numerical value or specific sub-range is expressly stated.

Various embodiments of the features of this disclosure are described herein. However, it should be understood that such embodiments are provided merely by way of example, and numerous variations, changes, and substitutions can occur according to those skilled in the art without departing from the scope of this disclosure. It should also be understood that various alternatives to the specific embodiments described herein are also within the scope of this disclosure.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features and advantages of the invention will be apparent from the following detailed description and figures, and from the claims.

### DESCRIPTION OF DRAWINGS

FIGS. 1A-1D show the synthesis and characterization of mPEG-b-p(HPMAm-Lacn) copolymer. FIG. 1A is a schematic showing a three-step synthetic scheme of mPEG-b-p(HPMAm-Lacn) copolymer. FIG. 1B shows a 1H NMR spectrum of HPMAm-Lacn monomer. FIG. 1C is a table showing the yield, GPC, and 1H NMR characterization of copolymer: the number average molecular weight (Mn), the weight average molecular weight (Mw), polydispersity (Mw/Mn). FIG. 1B shows a 1H NMR spectrum of mPEG-b-p(HPMAm-Lacn) copolymer.

FIG. 2A is a 1H NMR spectrum of gelatin and GelMA. FIG. 2B is a 1H NMR spectrum of HAGM.

FIGS. 3A-3D show micelle characterization, drug information and the schematic formation of drug loaded micelle. FIG. 3A is a table including the size (nm), PDI, and surface charge (mV) characterization of unloaded mPEG-b-p(HPMAm-Lacn) MCs. FIG. 3B is a representative TEM image of unloaded MCs (scale bar, 100 nm). FIG. 3C is a photographic image showing the physical appearance of LE loaded MCs (copolymer/drug ratio (w/w)=10:1). FIG. 3D is a table including the physical properties (solubility in water, lipophilicity Log P, H-bond donor, and acceptor) and structures of three corticosteroids: LE, PA, and DEX. FIG. 3E is a schematic of LE loaded MCs with drug and copolymer interaction shown in the box.

FIGS. 4A-4B show drug loaded MC characterization. FIG. 4A is a graph showing the drug (LE, PA and DEX) concentrations in 1 mL of MC solution with different initial copolymer/drug ratios (w/w, 10:0.25, 10:0.5, 10:1 and 10:2). FIG. 4B is a graph showing encapsulation efficiency (EE %) for LE, PA, and DEX with different initial copolymer/drug ratios (w/w, 10:0.25, 10:0.5, 10:1 and 10:2). FIGS. 4C-G are graphs showing the size in nanometer (nm) measurements (FIG. 4C), polydispersity index (PDI) (FIG. 4D), surface charge in millivolts (mV) (FIG. 4E), EE % (FIG. 4F), and loading capacity percentage (LC %) (FIG. 4G), respectively, for LE, PA, and DEX loaded MCs at the initial copolymer/drug ratio of 10:1. FIG. 4H are photographic images showing the appearance of LE loaded MCs in the dialysis bag immersed in the releasing medium of 2% Triton X-100 in DPBS at 37° C. on day 1, 3 and 5. FIG. 4I is a graph showing the cumulative release of LE, PA and DEX from mPEG-b-p(HPMAm-Lacn) MCs at 37° C. in the releasing medium of 2% Triton X-100 in DPBS measured by HPLC at different time points (30 min, 2 h, 1 day, 3 days, 5 days, 7 days and 10 days). Data are represented as means±SD (*P<0.1, **P<0.01, ***P<0.001, ****P<0.0001, n=3).

FIGS. 5A-5H show GelPatch formation and characterization. FIG. 5A is schematic of photocrosslinking of GelPatch+MCLE prepolymer solutions with the PI solution (Eosin Y, TEA and VC) and LE eluting from the crosslinked cylindrical hydrogel. FIGS. 5B-5F are graphs showing compression modulus (FIG. 5B), ultimate strain (FIG. 5C), ultimate stress (FIG. 5D), swelling ratio at 37° C. in DPBS after 24 h (FIG. 5E) and burst pressure of GelPatch, GelPatch+LE and GelPatch+MCLE fabricated using 7% GelMA and 3% HAGM (w/v) with 4-min photocrosslinking time (FIG. 5F), respectively. FIGS. 5G-5H are graphs showing in vitro release profiles of LE in 2% Triton X-100 releasing medium (FIG. 5G) and in vitro release profiles of LE (FIG. 511) in the presence of 5 μg/mL of collagenase and 5 μg/mL of hyaluronidase in 2% Triton X-100 releasing medium, respectively, from GelPatch+LE and GelPatch+MCLE at 37° C. in 15 days. All hydrogels were polymerized by using 0.5 mM Eosin Y, 1.875% (w/v) TEA and 1.25% (w/v) VC in PBS. Data are represented as means±SD (*P<0.1, **P<0.01, ****P<0.0001, n=3).

FIGS. 6A-6D show the in vitro biocompatibility of GelPatch and GelPatch+MC. FIG. 6A is a group of representative LIVE/DEAD images from hTCEpi cells seeded on hydrogels on day 1 and 3 (scale bar, 100 μm). FIG. 6B is a graph showing the quantification of cell viability on GelPatch and GelPatch+MC after 1 and 3 days of culture. FIG. 6C is a group of representative Actin/DAPI images from hTCEpi cells seeded on hydrogels on day 1 and 3 (scale bar, 100 μm). FIG. 6D is a graph showing the quantification of metabolic activity of hTCEpi cells seeded on GelPatch and GelPatch+MC after 1 and 3 days. Data are represented as means±SD (***P<0.001, ****P<0.0001, n≥3).

FIGS. 7A-7E show the in vivo biocompatibility and biodegradability of GelPatch and GelPatch+MC using a rat subcutaneous model. FIG. 7A is a group of hematoxylin and eosin (H&E) staining images, FIG. 7B is a group of Masson's Trichrome (MT) staining images, and FIG. 7C is a group of immunofluorescence (IF) staining images of GelPatch and GelPatch+MC sections (hydrogels with the surrounding tissue) after 7 and 28 days of implantation. FIG. 7D is a group of representative images of GelPatch and GelPatch+MC hydrogels before implantation (day 0), on day 7 and 28 post-implantation. FIG. 7E is a graph showing the in vivo biodegradation of hydrogels on day 7 and 28 post-implantation (scale bar, 100 μm). Data are represented as means±SD.

### DETAILED DESCRIPTION

The compositions described herein include biocompatible, photocrosslinkable gelatin-based adhesive hydrogels including one or more polymeric micelles. In some examples, the compositions described herein are used for targeted ocular drug delivery. Methods of using and/or preparing these compositions are also provided herein. Some embodiments of the compositions and methods described herein may provide one or more of the following advantages.

Certain embodiments of the present disclosure include biocompatible, photocrosslinkable gelatin-based adhesive hydrogels. As discussed above, there is currently an unmet need for a localized, noninvasive, and efficient ocular drug delivery vehicle that could have a high patient compliance. The compositions and methods of the present disclosure address this need. For example, in some embodiments, the compositions and methods described herein can achieve a sustained drug release profile for an extended period of time (e.g., about 5 to about 15 days). Furthermore, the compositions of the disclosure may facilitate drug penetration through the structural barriers of ocular tissues by adhering to the ocular surface and providing a sustained release of drugs directly to the injured sites. Thus, in some embodiments, a unique property of the compositions and methods described herein is its ability to provide localized and sustained delivery of one or more therapeutic agents directly in and/or onto the eye. Furthermore, in some embodiments, the compositions and methods described herein enable the delivery of hydrophobic therapeutic agents that can be encapsulated in the micelles.

Some embodiments described herein may provide a non-invasive, adhesive, and biocompatible drug-eluting patch that can provide lower dosage requirements to ensure better patient compliance. Thus, in some embodiments, the compositions and methods of the disclosure may offer a better alternative to current treatments, which are often invasive (e.g., injecting a therapeutic into the eye) or have low patient compliance (e.g., require a patient to self-administer one or more eye drops multiple times a day for an extended period of time).

Some embodiments described herein may provide site-targeted delivery to the eye. For example, systemic administration routes may require a large dose in order to achieve a satisfactory drug concentration at the ocular tissue, which can lead to off-target systemic side effects. On the other hand, local drug delivery such as the conventional topical administration (eye drops or ointments) have extremely low bioavailability (e.g., of about less than 5%) due to corneal epithelium barrier and fast clearance by tear film and blinking. As a result, repetitive drug applications may be required, which may induce ocular hypertension and are also associated with poor patient compliance. Furthermore, in some embodiments, the methods and compositions of the disclosure may provide high drug loading (e.g., hydrogel compositions including micelles that have a high drug-loading efficiency). Thus, in some embodiments, the compositions and methods of the disclosure may require lower doses and/or reduced number of applications to the eye while having a higher bioavailability.

Some embodiments described herein may provide optimum optical properties (e.g., optical transparency and wettability). In some embodiments, the compositions and methods of the disclosure may further provide patients with a drug delivery system that does not induce irritation or further inflammation or cause pain upon its application, thereby increasing patient compliance. Thus, in some embodiments, the compositions and methods of the disclosure may be well suited to be applied to the eye for extended periods of time.

Some embodiments described herein may provide a minimally invasive drug delivery vehicle and is not amenable to be removed by uncontrolled movements of the eye (e.g., uncontrolled retraction from the cornea and sclera), which could potentially cause leakage of the therapeutic agent being delivered. Thus, in some embodiments, the compositions and methods of the disclosure may provide a stable release of a therapeutic agent to a desired tissue (e.g., an optical tissue).

Some embodiments described herein may provide a composition that is biocompatible and biodegradable. For example, the compositions described herein were shown to have a high cell viability, cell adherence, and cell growth in vitro (see, e.g., Example 10). Furthermore, subcutaneous implantation of the micelle-loaded hydrogel composition in rats further confirmed its in vivo biocompatibility and appropriate stability throughout 28 days see, e.g., Example 11).

Some embodiments described herein may provide a composition that is easily applied to a tissue. For example, the precursor hydrogel composition loaded with micelles does not require a user to mix various components. Furthermore, the precursor hydrogel composition loaded with micelles does not have a limited time window (e.g., less than about 30 seconds) within which a user must apply the composition to a tissue before an undesirable change to the composition occurs (e.g., unwanted solidification, unwanted separation of components, or the like). Thus, in some embodiments, the compositions and methods of the disclosure provide a user with an efficient method to seal and deliver a therapeutic agent to a tissue.

Some embodiments described herein may provide a composition that may have a quick gelation time while retaining a high adhesive strength, especially when in contact with wet surfaces. For example, the precursor hydrogel composition loaded with micelles may solidify within a few minutes (e.g., less than about 4 minutes) upon exposure to a visible light. Furthermore, the precursor hydrogel composition loaded with micelles may exhibit a high adhesive strength (e.g., an ultimate stress of about 800 kPa or more) even when in contact with a wet surface.

Some embodiments described herein may provide a composition that may have batch-to-batch reproducibility. For example, the precursor hydrogel composition loaded with micelles may advantageously lack batch-to-batch product variations and the potential presence of viral contamination that is often present in naturally-derived products (e.g., that are not chemically modified). Thus, the in some embodiments, the compositions and methods of the disclosure may provide a safe and reproducible composition that is amenable to be produced at a commercial scale.

Some embodiments described herein may provide a composition that may have tunable properties. For example, the compositions described herein may be optimized at both liquid and solid states for high ocular retention upon instillation, post-crosslinking adhesion, swelling ratio, adhesive strength, and other mechanical properties while retaining high in vitro and in vivo cytocompatibility. The compositions of the disclosure may be easily optimized by varying the ratio of its components (e.g., HAGM, GelMA, photoinitiator, and micelles). Thus, the in some embodiments, the compositions and methods of the disclosure may provide a flexible drug delivery platform that can be optimized for various tissue types and injuries.

Some embodiments described herein provide a block co-polymer that is biodegradable. Backbone hydrolysis-based degradation of amphiphilic block copolymers, such as in PEG-b-polyester block copolymers, can facilitate the drug release due to the dramatic change in polarity of the core-forming block. The degradation mechanism and its profile can be modified by changing the feed ratio of N-(2 hydroxypropyl)methacrylamide-oligolactate (HPMAm-Lacn) monomers with different lactate chains lengths. Changing the degradation rate may provide another method to control and fine tune the drug release profile.

### Compositions

The present disclosure features biocompatible and adhesive compositions (e.g., hydrogels) that can include one or more micelles (MCs), a chemically modified gelatin (e.g., gelatin methacryloyl (GelMA)), a chemically modified hyaluronic acid (e.g., hyaluronic acid-glycidyl methacrylate (HAGM)), and a photoinitiator. In some embodiments, the compositions of the disclosure are drug-eluting hydrogels or “patches” that may facilitate the penetration of a drug through the structural barriers of tissues (e.g., ocular tissues) upon application and adhesion. For example, the compositions of the disclosure can be adhered to an ocular surface and provide a sustained release of drugs directly to an injured and/or disease site in an eye of a patient. To this end, the compositions described herein can include solubilized therapeutic agents in polymeric MCs, which can further protect the therapeutic agents and help provide a sustained release of the therapeutic agent to a tissue. In some embodiments, these optimized MCs formulations can be loaded within the compositions (e.g., GelPatch) of the disclosure.

Polymeric micelles can be used as a drug delivery vehicle within the hydrogel compositions to deliver hydrophobic therapeutic agents. For example, polymeric micelles composed of a hydrophilic shell and a hydrophobic core are amenable to encapsulate and deliver hydrophobic drugs. In some embodiments, the compositions described herein are a polymeric MC-based ocular drug delivery system that can achieve a sustained release of a therapeutic agent. In some embodiments, the polymeric micelles are formed by using a diblock copolymer, triblock copolymer, a graft copolymer, an amphiphilic copolymer, a multiblock copolymer, or any combination thereof. In some embodiments, the polymeric micelles are formed by using a diblock copolymer. In some embodiments, the micelles are block copolymer micelles comprising a hydrophobic core and a hydrophilic shell. In some embodiments, the block copolymer is poly(ethylene glycol)-b-poly(N-(2-hydroxypropyl) methacrylamide-oligolactate) (mPEG-b-p(HPMAm-Lacn)). In some embodiments, the micelles are biodegradable. In some embodiments, the micelles are biocompatible. The micelles can be formed by self-assembly via solvent evaporation methods, as described in Example 1, or by any other suitable methods.

As used herein, the term “self-assembly” refers to a process of spontaneous organization of components of a higher order structure by reliance on the attraction of the components for each other, and without chemical bond formation between the components. For example, polymer chains may interact with each other via any one of hydrophobic forces, hydrogen bonding, Van der Waals interaction, electrostatic forces, or polymer chain entanglement, induced on the polymer chains, such that hydrophilic regions of the polymer may aggregate or coagulate in an aqueous medium resulting in the hydrophobic regions of the polymer being sequestered at a center or core, thereby forming micelles.

Skilled practitioners will appreciate that any number of known materials can be used to prepare micelles, including, but not limited to, diblock polymers, triblock polymers, and graft copolymers. Additional polymers that can be used to generate the micelles to be dispersed in the composition include, but are not limited to, poly(D,L-lactic-co-hydroxymethyl glycolic acid) (PLHMGA), methoxy poly

(ethylene glycol)-b-(N-(2-benzoyloxypropyl)methacrylamide) (mPEG-b-p(HPMAm-Bz)), polyethylene glycol-poly lactic acid-co-glycolic acid (PEG-PLGA), other Amphiphilic AB diblock and BAB triblock copolymers

consisting of poly(ethylene glycol) (PEG) as hydrophilic A block and thermosensitive poly(N-isopropylacrylamide) (pNIPAm) B block and poly-(d,l-lactide) as hydrophobic B block (PDLLA), and polyethylene glycol (PEG) as hydrophilic A block and of semi-crystalline poly-(1-lactide) (PLLA) as hydrophobic B block.

In some embodiments, the polymer can have a chain length of about 1 kDa to about 80 kDa (e.g., about 1 kDa to about 20 kDa, about 5 kDa to about 20 kDa, about 10 kDa to about 20 kDa, about 15 kDa to about 20 kDa, about 20 kDa to about 25 kDa, about 20 kDa to about 30 kDa, about 20 kDa to about 35 kDa, about 20 kDa to about 40 kDa, about 20 kDa to about 45 kDa, about 20 kDa to about 50 kDa, about 20 kDa to about 55 kDa, about 20 kDa to about 60 kDa, about 20 kDa to about 65 kDa, about 20 kDa to about 70 kDa, about 20 kDa to about 75 kDa, or about 20 kDa to about 80 kDa). In some embodiments, the polymer can have a chain length of about 20 kDa.

In some embodiments, the composition includes micelles having a diameter ranging from about 50 nm to about 150 nm or more (e.g., about 50 nm to about 100 nm, about 50 nm to about 105 nm, about 50 nm to about 110 nm, about 50 nm to about 115 nm, about 50 nm to about 120 nm, about 50 nm to about 125 nm, about 50 nm to about 150 nm, about 75 nm to about 100 nm, about 75 nm to about 105 nm, about 75 nm to about 110 nm, about 75 nm to about 115 nm, about 75 nm to about 120 nm, about 75 nm to about 125 nm, about 75 nm to about 150 nm, about 90 nm to about 100 nm, about 90 nm to about 105 nm, about 90 nm to about 110 nm, about 90 nm to about 115 nm, about 90 nm to about 120 nm, about 90 nm to about 125 nm, about 90 nm to about 150 nm, about 100 nm to about 105 nm, about 100 nm to about 110 nm, about 105 nm to about 115 nm, about 105 nm to about 120 nm, about 105 nm to about 125 nm, about 105 nm to about 150 nm, about 110 nm to about 115 nm, about 110 nm to about 120 nm, about 110 nm to about 125 nm, about 110 nm to about 150 nm, about 115 nm to about 120 nm, about 115 nm to about 125 nm, about 115 nm to about 150 nm, about 120 nm to about 125 nm, about 120 nm to about 150 nm, or more). In some embodiments, the micelles have a diameter of about 100 nm. In some embodiments, the micelles have a diameter of about 118 nm. In some embodiments, the micelles have a diameter of about 120 nm. In some embodiments, the micelles have a diameter of about 110 nm. In some embodiments, the micelles have a diameter of about 84 nm.

The polydispersity index (PDI) is a measure of the heterogeneity of a sample based on size. For example, the PDI describes the width or spread of a particle size distribution. The PDI value is dimensionless and may vary from 0 to 1, where particles (e.g., micelles) with PDI values approaching 0 (e.g., less than about 0.1) may imply a monodisperse particle size distribution, and particles (e.g., micelles) with PDI values approaching 1 may imply a more polydisperse particle size distribution. In some embodiments, the composition includes micelles having a polydispersity index ranging from about at least 0.01 to about 0.2 (e.g., about 0.01 to about 0.08, about 0.01 to about 0.9, about 0.01 to about 0.1, about 0.01 to about 0.2, about 0.02 to about 0.08, about 0.02 to about 0.9, about 0.02 to about 0.1, about 0.02 to about 0.2, about 0.03 to about 0.08, about 0.03 to about 0.9, about 0.03 to about 0.1, about 0.03 to about 0.2, about 0.04 to about 0.08, about 0.04 to about 0.9, about 0.04 to about 0.1, about 0.04 to about 0.2, about 0.05 to about 0.08, about 0.05 to about 0.9, about 0.05 to about 0.1, about 0.05 to about 0.2, about 0.06 to about 0.08, about 0.06 to about 0.9, about 0.06 to about 0.1, about 0.06 to about 0.2, about 0.07 to about 0.08, about 0.07 to about 0.9, about 0.07 to about 0.1, about 0.07 to about 0.2, about 0.08 to about 0.9, about 0.08 to about 0.1, about 0.08 to about 0.2, about 0.09 to about 0.1, about 0.09 to about 0.2, or about 0.1 to about 0.2). In some embodiments, the micelles have a polydispersity index of about 0.09.

In some embodiments, the composition includes micelles having a net neutral surface charge density. Micelles having a net neutral surface charge density may be desirable to enable a slow degradation rate and serve as sustained drug release carriers. In some embodiments, the composition includes micelles having a surface charge of about −0.8 millivolts (mV) to about 0.2 mV (e.g., about −0.8 mV to about −0.2 mV, about −0.8 mV to about −0.1 mV, about −0.8 mV to about 0 mV, about −0.8 mV to about 0.05 mV, about −0.8 mV to about 0.1 mV, about −0.8 mV to about 0.2 mV, about −0.7 mV to about −0.2 mV, about −0.7 mV to about −0.1 mV, about −0.7 mV to about 0 mV, about −0.7 mV to about 0.05 mV, about −0.7 mV to about 0.1 mV, about −0.7 mV to about 0.2 mV, about −0.6 mV to about −0.2 mV, about −0.6 mV to about −0.1 mV, about −0.6 mV to about 0 mV, about −0.6 mV to about 0.05 mV, about −0.6 mV to about 0.1 mV, about −0.6 mV to about 0.2 mV, about −0.5 mV to about −0.2 mV, about −0.5 mV to about −0.1 mV, about −0.5 mV to about 0 mV, about −0.5 mV to about 0.05 mV, about −0.5 mV to about 0.1 mV, about −0.5 mV to about 0.2 mV, about −0.4 mV to about −0.2 mV, about −0.4 mV to about −0.1 mV, about −0.4 mV to about 0 mV, about −0.4 mV to about 0.05 mV, about −0.4 mV to about 0.1 mV, about −0.4 mV to about 0.2 mV, about −0.3 mV to about −0.2 mV, about −0.3 mV to about −0.1 mV, about −0.3 mV to about 0 mV, about −0.3 mV to about 0.05 mV, about −0.3 mV to about 0.1 mV, about −0.3 mV to about 0.2 mV, about −0.2 mV to about −0.1 mV, about −0.2 mV to about 0 mV, about −0.2 mV to about 0.05 mV, about −0.2 mV to about 0.1 mV, about −0.2 mV to about 0.2 mV, about −0.1 mV to about 0 mV, about −0.1 mV to about 0.05 mV, about −0.1 mV to about 0.1 mV, or about −0.1 mV to about 0.2 mV). In some embodiments, the micelles have a surface charge of about −0.2 mV.

In some embodiments, the unloaded micelles (e.g., micelles that are not encapsulating or otherwise loaded with a therapeutic agent) have a surface charge of about −7 millivolts (mV) to about −1 mV (e.g., about −7 mV to about −3 mV, about −7 mV to about −2 mV, about −7 mV to about −1 mV, about −6 mV to about −3 mV, about −6 mV to about −2 mV, about −6 mV to about −1 mV, about −5 mV to about −3 mV, about −5 mV to about −2 mV, about −5 mV to about −1 mV, about −4 mV to about −3 mV, about −4 mV to about −2 mV, or about −4 mV to about −1 mV). In some embodiments, the unloaded micelles have a surface charge of about −5 mV.

In some embodiments, the composition includes micelles at a concentration of about 5 mg/ml to about 30 mg/ml (e.g., about 5 mg/ml to about 10 mg/ml, 5 mg/ml to about 15 mg/ml, 5 mg/ml to about 20 mg/ml, 5 mg/ml to about 25 mg/ml, 5 mg/ml to about 30 mg/ml, about 10 mg/ml to about 15 mg/ml, 10 mg/ml to about 20 mg/ml, 10 mg/ml to about 25 mg/ml, 10 mg/ml to about 30 mg/ml, 15 mg/ml to about 20 mg/ml, 15 mg/ml to about 25 mg/ml, 15 mg/ml to about 30 mg/ml, 20 mg/ml to about 25 mg/ml, 20 mg/ml to about 30 mg/ml, or 25 mg/ml to about 30 mg/ml).

In some embodiments, the compositions described herein include a therapeutic agent (e.g., as a drug delivery payload). In some embodiments, the micelles include one or more therapeutic agents. In some embodiments, the therapeutic agents are encapsulated in, carried by, or otherwise loaded in or on the micelles and are not free within the composition (e.g., external to the micelles). In some embodiments, one or more therapeutic agents are dispersed within the composition but not loaded in or on the micelles. In other words, the therapeutic agents may be dispersed within the composition external to the micelles. In some embodiments, a first therapeutic agent is encapsulated in, carried by, or otherwise loaded in or on the micelles, and a second therapeutic agent is outside the micelles within the same composition. In some embodiments, the first and second therapeutic agents are the same. In some embodiments, the first and second therapeutic agents are the different. In some embodiments, the therapeutic agents are dispersed, embedded, suspended, and/or mixed within the composition.

In some embodiments, the micelles encapsulate a therapeutic agent at an encapsulation efficiency ranging from about 20% to about 95% (e.g., about 20% to about 25%, about 20% to about 30%, about 20% to about 40%, about 20% to about 50%, about 20% to about 55%, about 20% to about 60%, about 20% to about 65%, about 20% to about 70%, about 20% to about 75%, about 20% to about 80%, about 20% to about 85%, about 20% to about 90%, about 20% to about 95%, about 25% to about 30%, about 50% to about 55%, about 50% to about 60%, about 55% to about 60%, about 65% to about 70%, about 65% to about 75%, about 65% to about 80%, about 65% to about 85%, or about 65% to about 95%).

In some embodiments, the amount of therapeutic agent loaded inside the micelles varies with different polymer/drug ratios (e.g., the ratio of polymer to therapeutic agent during the preparation of micelles). In some embodiments, the micelles include a polymer/drug ratio ranging from about 10:0.1 to about 10:3 (w/w) (e.g., about 10:0.1 to about 10:0.25, about 10:0.1 to about 10:0.5, about 10:0.1 to about 10:1, about 10:0.1 to about 10:2, about 10:0.1 to about 10:3, about 10:0.25 to about 10:0.5, about 10:0.25 to about 10:1, about 10:0.25 to about 10:2, about 10:0.25 to about 10:3, about 10:0.5 to about 10:1, about 10:0.5 to about 10:2, about 10:0.5 to about 10:3, about 10:1 to about 10:2, about 10:1 to about 10:3, or about 10:2 to about 10:3).

In some embodiments, the therapeutic agent is a hydrophobic therapeutic agent. In some embodiments, the therapeutic agent is an anti-inflammatory therapeutic agent. In some embodiments, the therapeutic agent is a corticosteroid. In some embodiments, the therapeutic agent is loteprednol etabonate (LE), prednisolone acetate (PA), dexamethasone (DEX), or any combination thereof. In some embodiments, the therapeutic agent is loteprednol etabonate (LE). Exemplary therapeutic agents for inclusion in the compositions include, but are not limited to, an antibacterial, an anti-fungal, an anti-viral, an anti-acanthamoebal, an anti-inflammatory, an immunosuppressive, an anti-glaucoma, an anti-VEGF, a growth factor, or any combination thereof.

In some embodiments, a composition that includes a therapeutic agent, has improved healing properties compared to a composition without a therapeutic agent (e.g., it can reduce the time that it takes for an ocular injury to heal when treated with the composition, as compared to treatment with other commercially available ocular sealants or to compositions that do not include a therapeutic agent, for example). In some embodiments, drug released by the composition can reduce the risk of inflammation and contamination following injury. In some embodiments, drug released by the composition can promote wound healing.

Non-limiting examples of suitable anti-inflammatory agents include a steroidal anti-inflammatory drug (e.g., prednisolone), a non-steroidal anti-inflammatory drug (e.g., bromfenac), an mTOR inhibitor, a calcineurin inhibitor, a synthetic or natural anti-inflammatory protein, antiproliferative drugs (e.g., dexamethasone, 5-fluorouracil, daunomycin, paclitaxel, curcumin, resveratrol, and mitomycin), methylprednisolone, prednisolone, hydrocortisone, fludrocortisone, prednisone, celecoxib, ketorolac, piroxicam, diclorofenac, ibuprofen, and ketoprofen, rapamycin, cyclosporin, and tacrolimus/FK-506.

In some embodiments, the growth factor is epithelial growth factor, fibroblast growth factor, nerve growth factor, hepatocyte growth factor, or any combination thereof. Further non-limiting examples of suitable growth factors include transforming growth factors (TGFs) (e.g., beta transforming growth factors such as, TGF-β1, TGF-β2, TGF-β3), fibroblast growth factors (FGFs), platelet derived growth factors (PDGFs), epidermal growth factors (EGFs), connective tissue activated peptides (CTAPs), osteogenic factors, bone morphogenetic proteins (e.g., BMP-1, BMP-2, BMP-3, BMP-4, BMP-5, BMP-6, BMP-7, BMP-8, BMP-9); heparin-binding growth factors (e.g., fibroblast growth factor (FGF), epidermal growth factor (EGF), insulin-like growth factor (IGF)), Inhibins (e.g., Inhibin A, Inhibin B), growth differentiating factors (for example, GDF-1), and Activins (e.g., Activin A, Activin B, Activin AB), and biologically active analogs, fragments, and derivatives of such growth factors.

In addition to the micelles, the compositions of the disclosure can include gelatin methacryloyl (GelMA), hyaluronic acid-glycidyl methacrylate (HAGM), and a visible light-activated photoinitiator. In some embodiments, the composition is a hydrogel having micelles suspended within its three-dimensional matrix. In some embodiments, the physical properties of hydrogels may be similar to native tissue, and hydrogels may be used to encapsulate therapeutic agents in the hydrogel matrix formed upon gelation. In some embodiments, the composition is an injectable hydrogel, which may be injected into a subject in need thereof.

Generally, a hydrogel may be formed by using at least one, or one or more types of hydrogel precursors, and setting or solidifying the one or more types of hydrogel precursors in an aqueous solution to form a three-dimensional network, wherein formation of the three-dimensional network may cause the one or more types of hydrogel precursors to gel. As used herein, the term “hydrogel precursor” refers to any chemical compound that may be used to form a hydrogel. Examples of hydrogel precursors include, but are not limited to, a natural polymer, a hydrophilic monomer, a hydrophilic polymer, a hydrophilic copolymer formed from a monomer and a polymer. In some embodiments, the hydrogel precursor includes a chemically-modified polymer. The chemically-modified polymer may form a three-dimensional network in an aqueous medium to form a hydrogel. In some embodiments, the chemically-modified polymer is GelMA and/or HAGM.

Hyaluronic acid (HA) is a viscoelastic and highly biocompatible glycosaminoglycan, that is naturally present in the cornea. HA is known to play a role in the regeneration and reconstruction of soft tissues. In some embodiments, a chemically modified HA can be included in the compositions of the present disclosure. In some embodiments, the chemically modified HA can be methacrylated hyaluronic acid or a photocrosslinkable derivative of HA. In some embodiments, methacrylation of HA can be performed by ring opening of the HA backbone reaction and a reversible transesterification reaction. In some embodiments, the methacrylated hyaluronic acid included in the composition is HAGM.

In some embodiments, the HAGM is present in the composition at a concentration of about 0.5% and about 5% weight per volume (w/v) (e.g., about 0.5% to about 5%, about 1% to about 5%, about 1.5% to about 5%, about 2% to about 5%, about 2.5% to about 5%, about 3% to about 5%, about 0.5% to about 3%, about 0.5% to about 4%, about 1% to about 3%, or about 2% to about 3% (w/v)). In some embodiments, the HAGM is present in the composition at a concentration 3% (w/v). In some embodiments, the HAGM is present in the composition at a concentration 0.5% (w/v).

Gelatin is a derivative from collagen, which is the main structural component of the cornea. Gelatin has strong adhesive properties to cells and tissue due to the presence of RGD motifs in gelatin, a denatured form of collagen that is chemically modified to form a light-activated precursor. In some embodiments, a chemically modified gelatin can be included in the compositions of the present disclosure. In some embodiments, the chemically modified gelatin can be modified with methacryloyl anhydride (MA) to form GelMA, a photocrosslinkable derivative of gelatin. In some embodiments, chemical modification of gelatin can be performed by a synthesis reaction of gelatin with methacrylic anhydride (MAA).

As described in Example 3 and Equation 4, the degree of methacrylation (DM) of GelMA can be defined as the ratio of methacrylate groups to the free amine groups in gelatin prior to the reaction. In some embodiments, the composition includes GelMA with a degree of methacrylation (e.g., methacryloyl functionalization) ranging from at least about 30% to about 85% (e.g., about 30% to about 65%, about 40% to about 65%, about 50% to about 65%, about 60% to about 65%, about 61% to about 65%, about 65% to about 70%, about 62% to about 68%, about 63% to about 67%, about 64% to about 66%, about 61% to about 70%, about 60% to about 75%, about 60% to about 80%, about 60% to about 85%, about 40% to about 70%, about 40% to about 75%, about 40% to about 80%, about 40% to about 85%, about 50% to about 70%, about 50% to about 75%, about 50% to about 80%, or about 50% to about 85%). In some embodiments, the composition includes GelMA with a degree of substitution of about 61%. In some embodiments, the GelMA includes methacrylamide substitution and methacrylate substitution. In some embodiments, the ratio of methacrylamide substitution to methacrylate substitution is between about 80:20 and 99:1. In some embodiments, the ratio of methacrylamide substitution to methacrylate substitution can range from 80:20 to 85:15, 85:25 to 90:10, 90:10 to 95:5, or 95:5 to 99:1.

In some embodiments, the concentration of GelMA in the composition can range from about 3% to about 14% (w/v) (e.g., about 3% to about 7%, about 3% to about 8%, about 30% to about 9%, about 30% to about 10%, about 30% to about 110%, about 30% to about 12%, about 3% to about 13%, about 3% to about 14%, about 4% to about 7%, about 4% to about 8%, about 4% to about 9%, about 4% to about 10%, about 4% to about 11%, about 4% to about 12%, about 4% to about 13%, about 4% to about 14%, about 5% to about 7%, about 5% to about 8%, about 5% to about 9%, about 5% to about 10%, about 5% to about 11%, about 5% to about 12%, about 5% to about 13%, about 5% to about 14%, about 6% to about 7%, about 6% to about 8%, about 6% to about 9%, about 6% to about 10%, about 6% to about 11%, about 6% to about 12%, about 6% to about 13%, about 6% to about 14%, about 7% to about 8%, about 7% to about 9%, about 7% to about 10%, about 7% to about 11%, about 7% to about 12%, about 7% to about 13%, or about 7% to about 14% (w/v)). In some embodiments, the composition includes GelMA at a concentration of about 7% (w/v). In some embodiments, the composition includes one or more polymeric micelles, GelMA at a concentration of about 7% (w/v), and HAGM at a concentration of about 3% (w/v).

In some embodiments, the compositions can include a photoinitiator that can be used to activate polymerization and solidification of the composition when it is in a non-solid (e.g., viscous liquid, gel, liquid, or solution) form. In some embodiments, exposing the composition to light activates the photoinitiator, triggering the formation of free-radicals, resulting in vinyl-bond crosslinking between methacrylate groups, and thus polymerization of the composition, which results in a physical change of the composition from a solvent form to a hydrogel form. In some embodiments, the composition is in a form of a solution. In some embodiments, the composition is in a form of a hydrogel.

Different types of light sources can be used to photo-crosslink the hydrogel precursor composition (e.g., the composition in a solution form). Non-limiting examples of light sources that can be used to polymerize the composition include visible light sources (e.g., white or blue light), ultraviolet light sources, near-infrared light sources, and fluorescent light sources. In some embodiments, the composition includes a visible light-activated photoinitiator that can be activated upon exposure of light having a wavelength between about 420 nanometers (nm) to 550 nm. In some embodiments, the visible light-activated photoinitiator can be activated upon exposure of light having a wavelength of about 460 nm. In some embodiments, the visible light-activated photoinitiator can be activated upon exposure of light having a wavelength ranging from about 400 nm to about 800 nm. In some embodiments, the visible light-activated photoinitiator can be activated upon exposure of light having a wavelength less than 800, 750, 700, 650, 600, 550, 500, 450, or 400 nm. In some embodiments, the visible light-activated photoinitiator can be activated upon exposure of light having a wavelength greater than 400, 450, 500, 550, or 600 nm.

In some embodiments, the photoinitiator includes a light-activated photoinitiator. In some embodiment, the light-activated photoinitiator includes an ultraviolet light-activated photoinitiator. In some embodiment, the light-activated photoinitiator includes a near-infrared (NIR) light-activated photoinitiator. In some embodiment, the light-activated photoinitiator includes a visible light-activated photoinitiator. In some embodiment, the visible light-activated photoinitiator includes Eosin Y, triethanolamine (TEA), N-vinylcaprolactam (VC), or any combination thereof. In some embodiments, the light-activated photoinitiator can include a blue light-activated photoinitiator. In some embodiments, the visible light-activated photoinitiator includes triethanolamine, N-vinylcaprolactam, riboflavin, 2-hydroxy-4′-(2-hydroxyethoxy)-2-methylpropiophenone, Eosin Y disodium salt, 4,6-trimethylbenzoylphosphinate, triethanol amine, dl-2,3-diketo-1,7,7-trimethylnorcamphane (CQ), 1-phenyl-1,2-propadione (PPD), 2,4,6-trimethylbenzoyl-diphenylphosphine oxide (TPO), bis(2,6-dichlorobenzoyl)-(4-propylphenyl)phosphine oxide, 4,4′-bis(dimethylamino)benzophenone, 4,4′-bis(diethylamino)benzophenone, 2-chlorothioxanthen-9-one, 4-(dimethylamino)benzophenone, phenanthrenequinone, ferrocene, diphenyl(2,4,6 trimethylbenzoyl)phosphine oxide/2-hydroxy-2-methylpropiophenone (50/50 blend), dibenzosuberenone, (benzene) tricarbonylchromium, resazurin, resorufin, benzoyltrimethylgermane, derivatives thereof, or any combination thereof. In some embodiments, the visible light-activated photoinitiator includes a mixture of triethanolamine, N-vinylcaprolactam, riboflavin, 2-hydroxy-4′-(2-hydroxyethoxy)-2-methylpropiophenone, and Eosin Y disodium salt. In some embodiments, the visible light-activated photoinitiator comprises a mixture of two or more elements selected from triethanolamine, N-vinylcaprolactam, riboflavin, 2-hydroxy-4′-(2-hydroxyethoxy)-2-methylpropiophenone, and Eosin Y disodium salt.

In some embodiments, at least two of the GelMA, the HAGM, the visible light-activated photoinitiator, and the one or more micelles are formulated in separate formulations. For example, in some embodiments, the micelles are formulated separately from the precursor hydrogel composition including GelMA, HAGM, and the visible light-activated photoinitiator. In some embodiments, the micelles are first formulated to encapsulate a therapeutic agent and then are mixed with the precursor hydrogel composition including GelMA, HAGM, and the visible light-activated photoinitiator.

The release kinetics of the composition including micelles loaded with a therapeutic agent can be controlled by adjusting the concentration of one or more of the polymers in the formulation (e.g., HAGM and/or GelMA). In some embodiments, the composition delivers at least about 50% to about 85% of the therapeutic agent from the micelles in about 1 day. In some embodiments, the composition exhibits a burst release within about 2 hours after application. In some embodiments, about 20% to about 50% (e.g., about 20% to about 24%, about 20% to about 40%, about 20% to about 50%, or more) of the therapeutic agent is released from the micelles and the composition within about 2 hours after application.

The incorporation of micelles within the hydrogel composition can enable solubilization of therapeutic agents and its sustained release within an extended time frame (e.g., about 15 days). In some embodiments, the incorporation of micelles into the hydrogel composition retards the release of the therapeutic agent and leads to an almost linear drug release profile over an extended time frame (e.g., about 15 days). In some embodiments, the composition has a sustained therapeutic release profile over a period of about 15 days at most. In some embodiments, about 95% to about 100% of the therapeutic agent is released within about 12 to about 15 days.

In some embodiments, the drug release profile can be controlled by increasing the density of crosslinking, which can have impact on release rate of a payload (e.g., small drug molecules). In some embodiments, the drug release profile can be decreased by increasing the density of crosslinking of the polymer(s) of the hydrogel compositions. In some embodiments, increasing the density of crosslinking can be controlled by increasing the photo-crosslinking time, increasing the photoinitiator composition, changing the GelMA/HAGM concentration, or any combination thereof. In some embodiments, since the release of small molecules can be controlled via diffusion, the higher the crosslinking density, the more steric hindrance and the slower the release of the small molecule from the any of the disclosed compositions.

In some embodiments, the drug release profile can be decreased by increasing the stiffness of crosslinking of the polymer(s) of the hydrogel compositions. Alternatively, in some embodiments, the drug release profile can be increased by decreasing the stiffness of crosslinking of the polymer(s) of the hydrogel compositions. In some embodiments, the drug release profile can be decreased by decreasing the swelling properties of the polymer(s) of the hydrogel compositions. Alternatively, in some embodiments, the drug release profile can be increased by increasing the swelling properties of the polymer(s) of the hydrogel compositions.

In some embodiments, the drug release profile can be controlled by the molecular structure of the polymer(s) of the hydrogel compositions. For example, in some embodiments, some blocks of polymer and/or protein chains of the hydrogel composition can form hydrophobic interactions with the payload (e.g., a drug molecule) and slow down the payload release.

In some embodiments, the drug release profile (e.g., of a hydrophobic drug molecule payload) can be controlled by the concentration of the polymer(s) of the hydrogel compositions due to the formation of a denser crosslinked matrix. For example, in some embodiments, a hydrogel composition having an increased polymeric concentration(s) can yield a denser, crosslinked matrix, thereby causing a slower drug release profile (e.g., of a hydrophobic drug molecule payload).

In some embodiments, modification of the drug-loaded particles' (e.g., drug-loaded micelles') properties, which are loaded inside the hydrogel, can impact overall drug release rate and/or profile. Non-limiting examples of such modifications include: the chain length of hydrophobic block (e.g., can range from about 2 kDa to about 20 kDa); crosslinking of the particles' (e.g., micelles') core, which can be achieved, for example, via the addition of methacrylamide as functional group (e.g., a functional side group); addition of benzene side chains at a hydrophobic block of a polymer used to synthesize micelles; modifying the degradation kinetics of the synthetic block co-polymer used to synthesize the micelles; or any combination thereof.

In some embodiments, the addition of benzene side chains can enable sustained release of drug molecules containing benzene rings in order to induce pi-pi interactions. In some embodiments, an example derivative polymer comprising benzene that can be used to synthesize micelles to be encapsulated within the hydrogel compositions disclosed herein is methoxy poly(ethylene glycol)-b-(N-(2-benzoyloxy propyl)methacrylamide) (mPEG-b-p(HPMAm-Bz)). In some embodiments, modifying the degradation kinetics of the synthetic block co-polymer can be influenced by the feed ratio of N-(2 hydroxypropyl)methacrylamide-oligolactates (HPMAm-Lacn) monomers having different lactate chains lengths.

In some embodiments, the drug release profile can be controlled by the molecular structure of the payload (e.g., a small molecule, a small drug molecule, a hydrophilic drug molecule, a hydrophobic drug molecule, or the like) itself. For example, in some embodiments, the payload can be an enantiomer, which may have the tendency to form a crystalline structure more than an amorphous structure, which can significantly impact its release profile. For example, in some embodiments, the payload having a crystalline structure may have a slower release profile than the payload having an amorphous structure. In some embodiments, the formulation process of the payload (e.g., a small molecule, a small drug molecule, a hydrophilic drug molecule, a hydrophobic drug molecule, or the like) can induce crystallization of the loaded drug molecules, thereby slowing the drug release profile. In some embodiments, the concentration (e.g., a crystallization concentration) of the payload (e.g., a small molecule, a small drug molecule, a hydrophilic drug molecule, a hydrophobic drug molecule, or the like) can induce crystallization of the loaded drug molecules, thereby slowing the drug release profile. In some embodiments, if the payload is a hydrophilic drug molecule, the drug release profile can be faster (e.g., due to faster diffusion out of the nanoparticle and/or hydrogel compositions).

In some embodiments, the composition further includes a pharmaceutically acceptable carrier. As used herein, the expression “pharmaceutically acceptable carrier” refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation and is compatible with administration to a subject, for example a human. It must also be suitable for use in contact with any tissues or organs with which it may come in contact, meaning that it must not carry a risk of toxicity, irritation, allergic response, immunogenicity, or any other complication that excessively outweighs its therapeutic benefits. Examples of pharmaceutically acceptable carriers include, but are not limited to, a solvent or dispersing medium containing, for example, water, pH buffered solutions (e.g., phosphate buffered saline (PBS), HEPES, TES, MOPS, etc.), isotonic saline, Ringer's solution, polyol (for example, glycerol, propylene glycol, liquid polyethylene glycol, and the like), alginic acid, ethyl alcohol, and suitable mixtures thereof. In some embodiments, the pharmaceutically acceptable carrier can be a pH buffered solution (e.g. PBS).

In some embodiments, the pharmaceutically acceptable carrier is a topical carrier. In some embodiments, the composition is formulated for topical use. In some embodiments, the composition is topically administered to a tissue (e.g., an ocular tissue) of a patient. In some embodiments, the composition can be applied to a tissue (e.g., an ocular tissue) for topical, targeted delivery of a therapeutic agent.

### Physical Properties of Compositions

The physical properties of the compositions of the disclosure, including but not limited to stiffness, elasticity, degradation rate, adhesion, and swelling, can be finely tuned by modulating the concentration of one or more of the polymers (e.g., HAGM and/or GelMA) and/or photoinitiator. In some embodiments, these physical properties can be altered by changing the density (e.g., the number) of functional groups (e.g., methacrylated group) per polymer chain (e.g., per molecule). For example, in some embodiments, different types of functional groups can be used to modify the polymers, such as methacrylate or glycidyl methacrylate, which can be used for photo-crosslinking properties, or polyelectrolytes, which can be used for charge-based interactions and coacervation. In some embodiments, these physical properties can also be altered by varying the molecular weight (e.g., the polymer chain length) of the polymers used to synthesize the hydrogel compositions disclosed herein. For example, in some embodiments, using a smaller chain length polymer can yield increase stiffness and/or decrease elasticity, thereby yielding a rigid and stiff hydrogel composition. On the other hand, in some embodiments, using a higher molecular weight polymer (with same density of existing functional groups) to synthesize the hydrogel compositions disclosed herein can decrease stiffness and/or increase elasticity, thereby yielding a flexible hydrogel composition. In some embodiments, these physical properties can also be altered by the type of drug molecule loaded in the hydrogel compositions. For example, in some embodiments, if the payload (e.g., a small molecule or a small drug molecule) tends to be crystalline, rather than being amorphous, the payload can form nano-distributed crystalline domains within the hydrogel, which can impact the release rate (e.g., it can decrease the drug release rate).

Alternatively, or in combination to the polymer concentration modulation, the physical properties of the sealant can also be finely tuned by controlling the light exposure time (e.g., the polymerization time). In some embodiments, the composition is exposed to a light source for about 4 minutes. In some embodiments, the composition is exposed to a light source for about a period ranging from about 15 seconds to 15 minutes. In some embodiments, the composition is exposed to a light source for about a period ranging from about 1 to 10 minutes. In some embodiments, the composition is exposed to a light source for about 30 seconds to 1 minute, 1 to 2 minutes, 2 to 3 minutes, 3 to 4 minutes, 4 to 5 minutes, 5 to 6 minutes, 6 to 7 minutes, 7 to 8 minutes, 8 to 9 minutes, or 9 to 10 minutes. In some embodiments, the composition is exposed to a light source for less than about 20, 15, 10, or 7 minutes. In some embodiments, the composition is exposed to a light source for more than about 10 seconds, 30 seconds, 1, 3, or 5 minutes.

Treatment of ocular penetrating injuries (e.g., lacerations) is very challenging due to leakage of intraocular fluid from the injury site. In some embodiments, the composition is a viscous gel that can retain its shape and/or consistency on an ocular injury site without running-off and is able to stop intraocular fluid leaking through the injury site. The viscosity of the composition is an important property that allows the sealant to have a good retention (e.g., no run-off) on the surface of a cornea (e.g., when treating a corneal injury). In some embodiments, once polymerized and solidified, the composition has a viscosity that is greater than the viscosity of the precursor composition prior to photo-crosslinking and solidification. In some embodiments, the precursor composition (e.g., the composition prior to photo-crosslinking and solidification) has a viscosity that is greater than the viscosity of water. In some embodiments, the precursor composition has a viscosity that is similar to the viscosity of toothpaste. In some embodiments, the composition has a viscosity ranging from about 0.5 Pascal-seconds (Pa·s) to about 300 Pa·s. In some embodiments, the composition has a viscosity of about 100 Pa·s at a low shear rate (e.g., at a shear rate of about 0.001 inverse seconds (s⁻¹) to 1 s⁻¹. In some embodiments, the composition has a shear stress ranging from about 1 to 10 Pa at a low shear rate (e.g., at a shear rate of about 0.001 to 0.1 s⁻¹. In some embodiments, the composition includes about 3% HAGM, and the precursor composition has a viscosity of between about 30 Pa·s to about 300 Pa·s at a low shear rate (e.g., at a shear rate of about 0.001 inverse seconds (s⁻¹) to 1 s⁻¹. In some embodiments, the viscosity is about 100 Pa·s. In some embodiments, the composition includes about 3% HAGM and about 4% GelMA, and the precursor composition has a viscosity of between about 30 Pa·s to about 300 Pa·s at a low shear rate (e.g., at a shear rate of about 0.001 inverse seconds (s⁻¹) to 1 s⁻¹. In some embodiments, the viscosity is about 100 Pa·s. In some embodiments, the composition includes about 3% HAGM, and the precursor composition has a shear stress ranging from about 0.1 to 10 Pa at a low shear rate (e.g., at a shear rate of about 0.001 to 0.1 s⁻¹). In some embodiments, the composition includes about 3% HAGM and about 4% GelMA, and the precursor composition has a shear stress ranging from about 0.1 to 10 Pa at a low shear rate (e.g., at a shear rate of about 0.001 to 0.1 s⁻¹).

Important mechanical properties of the composition include compression modulus, ultimate stress (or ultimate tensile strength), extensibility, and swelling ratio. In some embodiments, the compression modulus can be varied based on changing the crosslinking density of the polymer(s) used to synthesize the hydrogel compositions of the disclosure. For example, in some embodiments, higher crosslinking can lead to rigid and stiff hydrogels, thereby increasing the compression modulus. In some embodiments, another factor that can affect the compression modulus is the concentration of the polymer(s) used to synthesize the hydrogel compositions of the disclosure. For example, in some embodiments, the higher the concentration of polymer, the higher the compression modulus can be. Thus, in some embodiments, increasing the concentration of one or more polymers used to synthesize the hydrogel compositions of the disclosure can increase the compression modulus of the hydrogel compositions disclosed herein.

In some embodiments, the composition has a compression modulus of about 15 kilopascals (kPa) to about 30 kPa (e.g., about 15 kPa to about 17 kPa, about 15 kPa to about 20 kPa, about 15 kPa to about 25 kPa, about 15 kPa to about 28 kPa, about 15 kPa to about 30 kPa, about 17 kPa to about 20 kPa, about 17 kPa to about 25 kPa, about 17 kPa to about 28 kPa, about 17 kPa to about 30 kPa, about 20 kPa to about 25 kPa, about 20 kPa to about 28 kPa, about 20 kPa to about 30 kPa, about 25 kPa to about 28 kPa, about 25 kPa to about 30 kPa, or about 28 kPa to about 30 kPa).

In some embodiments, the composition has an ultimate stress of about 350 kilopascals (kPa) to about 850 kPa (e.g., about 350 kPa to about 395 kPa, about 350 kPa to about 400 kPa, about 350 kPa to about 450 kPa, about 350 kPa to about 500 kPa, about 350 kPa to about 550 kPa, about 350 kPa to about 600 kPa, about 350 kPa to about 650 kPa, about 350 kPa to about 650 kPa, about 350 kPa to about 700 kPa, about 350 kPa to about 750 kPa, about 350 kPa to about 800 kPa, about 350 kPa to about 825 kPa, about 350 kPa to about 850 kPa, about 395 kPa to about 400 kPa, about 395 kPa to about 450 kPa, about 395 kPa to about 500 kPa, about 395 kPa to about 550 kPa, about 395 kPa to about 600 kPa, about 395 kPa to about 650 kPa, about 395 kPa to about 650 kPa, about 395 kPa to about 700 kPa, about 395 kPa to about 750 kPa, about 395 kPa to about 800 kPa, about 395 kPa to about 825 kPa, about 395 kPa to about 850 kPa, about 400 kPa to about 450 kPa, about 400 kPa to about 500 kPa, about 400 kPa to about 550 kPa, about 400 kPa to about 600 kPa, about 400 kPa to about 650 kPa, about 400 kPa to about 650 kPa, about 400 kPa to about 700 kPa, about 400 kPa to about 750 kPa, about 400 kPa to about 800 kPa, about 400 kPa to about 825 kPa, about 400 kPa to about 850 kPa, about 450 kPa to about 500 kPa, about 450 kPa to about 550 kPa, about 450 kPa to about 600 kPa, about 450 kPa to about 650 kPa, about 450 kPa to about 650 kPa, about 450 kPa to about 700 kPa, about 450 kPa to about 750 kPa, about 450 kPa to about 800 kPa, about 450 kPa to about 825 kPa, about 450 kPa to about 850 kPa, about 500 kPa to about 600 kPa, about 500 kPa to about 650 kPa, about 500 kPa to about 650 kPa, about 500 kPa to about 700 kPa, about 500 kPa to about 750 kPa, about 500 kPa to about 800 kPa, about 500 kPa to about 825 kPa, about 500 kPa to about 850 kPa, about 600 kPa to about 650 kPa, about 600 kPa to about 650 kPa, about 600 kPa to about 700 kPa, about 600 kPa to about 750 kPa, about 600 kPa to about 800 kPa, about 600 kPa to about 825 kPa, about 600 kPa to about 850 kPa, about 650 kPa to about 700 kPa, about 650 kPa to about 750 kPa, about 650 kPa to about 800 kPa, about 650 kPa to about 825 kPa, about 650 kPa to about 850 kPa, about 700 kPa to about 750 kPa, about 700 kPa to about 800 kPa, about 700 kPa to about 825 kPa, about 700 kPa to about 850 kPa, about 750 kPa to about 800 kPa, about 750 kPa to about 825 kPa, about 750 kPa to about 850 kPa, about 800 kPa to about 825 kPa, about 800 kPa to about 850 kPa, about 825 kPa to about 850 kPa).

In some embodiments, the composition has an extensibility of about 40%. In some embodiments, the composition has an extensibility ranging from between about 30% to about 60%. In some embodiments, the composition has an extensibility ranging from between about 40% to about 50%.

In some embodiments, the composition is a hydrogel. A hydrogel includes a polymer network filled with an interstitial solvent (e.g., a fluid) which may include water. A hydrogel can change its volume by absorbing a solvent (e.g., when it swells) or expelling a solvent. The swelling ratio of a hydrogel is defined as the fractional increase in the weight of the hydrogel due to water absorption. Typically, the swelling ratio depends on both the polymer/solvent and the elasticity of the polymer. If the polymer is too stiff or the affinity is too low, then the swelling is low or weak. In contrast, low elasticity and high affinity favor high swelling.

In some embodiments, the composition has a swelling ratio ranging from about 15% to about 20% (e.g., about 15% to about 16%, about 15% to about 17%, about 15% to about 18%, about 15% to about 19%, about 15% to about 20%, about 16% to about 19%, about 17% to about 20%, about 18% to about 20%, or about 15% to about 19%). In some embodiments, the composition has a short-term swelling ratio (e.g., a swelling ratio measured for a period of about 1 to 6 hours) of about 15%. In some embodiments, the composition has a short-term swelling ratio (e.g., a swelling ratio measured for a period of about 1 to 6 hours) of between about 15% to about 20%. In some embodiments, the composition has a mid-term swelling ratio (e.g., a swelling ratio measured for a period of about 1 to 3 days) of about 15%. In some embodiments, the composition has a mid-term swelling ratio (e.g., a swelling ratio measured for a period of about 1 to 3 days) of between about 15% to about 20%. In some embodiments, the composition has a long-term swelling ratio (e.g., a swelling ratio measured for a period of about 1 to 4 weeks) of about 15%. In some embodiments, the composition has a long-term swelling ratio (e.g., a swelling ratio measured for a period of about 1 to 4 weeks) of between about 15% to about 20%.

In some embodiments, the composition has a water content of about 94% or more. In some embodiments, the composition has a water content ranging from about 94% to about 97%. In some embodiments, the composition has a water content ranging from about 94% to about 95%.

The degradation rate of the composition can be controlled based on the concentration of one or more polymers added (e.g., HAGM and/or GelMA). In some embodiments, the composition has a degradation rate of about 35 days. In some embodiments, the composition has a degradation rate ranging from about 1 day to about 40 days. In some embodiments, the composition has a degradation rate ranging from about 1 to about 5 days, about 5 to about 10 days, about 10 to about 15 days, about 15 to about 20 days, about 20 to about 25 days, about 25 to about 30 days, about 30 to about 35 days, or about 35 to about 40 days. In some embodiments, the composition has a degradation rate of less than about 80, about 60, about 55, about 50, about 45, about 40, about 35, or about 30 days. In some embodiments, the composition has a degradation rate more than about 1, about 5, about 7, about 10, about 14, about 21, about 25, about 30, about 35, or about 40 days.

In some embodiments, the composition has high adhesive properties, especially in wet environments. Typically, to measure the adhesive strength of an optical sealant, an in vitro burst pressure test can be conducted in which a clinically representative incision is made in an ex vivo animal eye (e.g., a porcine eye). An infusion cannula can be placed inside the eye in order to reproduce the physiologic intraocular pressure. Once the incision is created, the optical sealant can be applied over the incision, and the intraocular pressure, required to rupture the sealant, can be measured. Such intraocular pressure can be defined as the “burst strength” or “burst pressure.”

In some embodiments, burst pressure can be determined by density and type of interactions between the hydrogel and collagen membrane, and the cohesiveness of the crosslinked hydrogel patch (e.g., the ability of the hydrogel to store deformation energy in an elastic manner). In some embodiments, burst pressure can be affected by both adhesive and cohesive properties of hydrogel compositions.

In some embodiments, the composition has a burst strength of about 10 kPa to about 30 kPa (e.g., about 10 kPa to about 11 kPa, about 10 kPa to about 12 kPa, about 10 kPa to about 13 kPa, about 10 kPa to about 15 kPa, about 10 kPa to about 20 kPa, about 10 kPa to about 25 kPa, about 10 kPa to about 30 kPa, about 11 kPa to about 12 kPa, about 11 kPa to about 13 kPa, about 11 kPa to about 15 kPa, about 11 kPa to about 20 kPa, about 11 kPa to about 25 kPa, about 11 kPa to about 30 kPa, about 12 kPa to about 15 kPa, about 12 kPa to about 20 kPa, about 12 kPa to about 25 kPa, about 12 kPa to about 30 kPa, about 13 kPa to about 15 kPa, about 13 kPa to about 20 kPa, about 13 kPa to about 25 kPa, or about 13 kPa to about 30 kPa).

### Methods of Treatment

The present disclosure presents methods of treating an ocular disease or an ocular injury (e.g., a corneal or scleral injury) in an eye of a subject. In some embodiments, the present disclosure presents compositions for use in the treatment an ocular disease or an ocular injury (e.g., a corneal or scleral injury) in an eye of a subject. The methods can include the steps of contacting the eye of the subject with any of the compositions disclosed herein and photo-crosslinking the composition by exposing the composition to a visible light.

In some embodiments, the compositions can be injected into the eye using a syringe. In some embodiments, the compositions can be applied onto a surface of the eye by using a syringe, a pipette (e.g., a Pasteur pipette), a brush, a dropper bottle or dropper tube configured to dispense a viscous fluid (e.g., having an aperture at a distal end that is large enough to dispense a viscous fluid), or any other suitable device or tool. In some embodiments, the precursor hydrogel compositions can be applied as a drop (e.g., when in a viscous, non-solid state) onto the eye without the need for an applicator. Exposure to visible light can permit crosslinking to provide an adhesive solid hydrogel with biomechanics analogous to the cornea. By adjusting the light exposure time, the polymerization of the adhesive compositions of the disclosure can be finely controlled, allowing for a precise application, as compared to commercially available ocular sealants.

In some embodiments, the methods include applying the composition to an applicator (e.g., a contact lens), contacting the applicator to the eye of the subject, and photo-crosslinking the composition. The first step can include filling the applicator with the composition. Once filled with the composition, the applicator is directly applied on the ocular injury, e.g., by using forceps. This applicator can allow a user (e.g., a clinician) to easily apply the precursor hydrogel composition on ocular injuries with only forceps. The applicator containing the composition can be inverted and placed on the surface of the eye of the subject having or suspected of having the ocular surface injury, without falling off or running off the surface of the applicator when inverted. Due to the high viscosity of the hydrogel precursor composition (e.g., a viscosity similar to the viscosity of toothpaste), leaking from the aqueous humor, for example, can be instantly halted when the applicator containing the composition is placed on the ocular surface. The precursor hydrogel composition can be applied on any size or shape of ocular injuries to stop leaks from aqueous humor. In some embodiments, between about 20 and 200 microliters (μL) of precursor hydrogel composition can be applied depending on the size and the shape of the ocular injury.

When the position of the applicator is satisfactory, the operator can initiate photo-crosslinking to solidify the composition by using a visible light source. In some embodiments, the composition can be photo-crosslinked by exposing the contact lens and the composition to a visible light. In some embodiments, the visible light has a wavelength of about 400 nanometers (nm) to 800 nm. After photo-crosslinking, the composition can become solid and transparent. Once the composition is photo-crosslinked, the applicator can be removed from the ocular surface (e.g., by using forceps).

The applicator can be any suitable contact lens; for example, a hard contact lens, a soft contact lens, or a non-contact lens applicator that will permit controlled application of the sealant on the tissue. Non-limiting examples of contact lens types include rigid gas-permeable lenses and bandage lenses. The applicator can be a contact lens of different materials, diameters, base curve radiuses, power in diopters and central thickness. The applicator can also have a smooth and regular surface that comes in contact with the ocular surface thereby, limiting patient discomfort and vision loss.

In some embodiments, the composition is used to prevent fluid leakage after cataract surgery. In some embodiments, the composition remains localized over the incision, injury, and/or laceration to seal the wound and form a surface barrier. In some embodiments, upon photo-crosslinking, the precursor composition becomes a solid and transparent hydrogel, forming a biocompatible and adhesive sealant on the ocular surface.

### Ocular Injuries

The present disclosure presents methods and compositions for treating ocular injuries (e.g., ocular surface injuries) in an eye of a subject. In some embodiments, the ocular injury is an injury or trauma resulting from an ocular surgery. In some embodiments, the compositions of the disclosure are used in post-surgical care. For example, the compositions may be administered to a patient after an ocular surgery to deliver a therapeutic agent (e.g., an anti-inflammatory agent or an antibiotic) that may be prescribed to minimize recovery time, prevent and/or treat inflammation caused by the surgical procedure, prevent and/or treat an ocular infection caused by the surgical procedure, or any combination thereof.

Ocular surface injuries can include conjunctival laceration, corneal perforation, scleral perforation, incisions due to ocular surgery (e.g., cataract surgery) or any combination thereof. In some embodiments, the ocular surface injury is a corneal or scleral injury. Conjunctival laceration may occur following blunt or penetrating trauma. Conjunctival laceration is characterized with chemosis and subconjunctival hemorrhage. In such cases, it is important to rule out underlying scleral perforation. The fundus should be examined for any retinal tear or intraocular foreign body. An ultrasound may be done for the posterior segment evaluation.

Corneal lacerations and perforations represent approximately 1 in 10 of ocular traumatic injuries presenting in an emergency medical setting. Corneal lacerations and perforations can include partial thickness lacerations and full thickness lacerations. In addition, adnexal injuries, scleral perforation, or a combination thereof may be involved with corneal laceration and perforations. The standard of care for a corneal perforation include the removal of any contaminants in the wound area, repair of the tear, and maintenance of the watertight integrity of the ocular globe. Corneal perforation may also be associated with or caused by insertion of a foreign body. In some embodiments, the corneal injury is a corneal full-thickness laceration or a corneal full-thickness perforation. In some embodiments, the ocular surface injury is a full-thickness laceration or a full-thickness perforation. In some embodiments, the ocular surface injury is a full-thickness laceration or surgical incision or a full-thickness perforation. For example, the majority of ocular surgeries that require entry into the eye (e.g., cataract surgery) involve a full-thickness incision through the cornea or sclera. Current management protocols for full thickness lacerations including scleral wounds often require the use of sutures.

The compositions of the disclosure can be used to treat ocular incisions or cuts or injuries having a length of less than about 1 mm to about 10 mm. In some embodiments, the compositions of the present disclosure can be used in the closure of full-thickness ocular defects and lacerations and in controlled and long-term drug elution. In some embodiments, indications can include post-operative applications of the biomaterial for drug elution in addition of closure of corneal ulcers, defects and perforations caused by a wide array of insults. The compositions of the disclosure can be applied both under “normal” (e.g., in-the-office or operating room) settings, or under emergency “in-in-field” settings. Various providers, physicians, and, in select cases, physician assistants and paramedics (e.g., in the combat theater) can apply the compositions described herein to seal the eye and elute drug(s) to heal defects. The compositions described herein can circumvent many cases of transplants and patch grafts for corneal melts and defects.

### Ocular Diseases

The present disclosure presents methods and compositions for treating ocular diseases in an eye of a subject. In some embodiments, the disease is an ocular anterior segment disease. Non-limiting examples of ocular anterior segment diseases include conjunctivitis (e.g., allergic, bacterial, and/or viral conjunctivitis), blepharitis, anterior segment dysgenesis, dry eye, meibomian gland dysfunction (MGD), keratoconus, uveitis, pterygium, a cataract, a herpes simplex and/or herpes zoster (shingles) infection, a chemical burn, keratoconus and other ectatic disorders (e.g., keratoglobus, pellucid marginal degeneration), Fuchs' endothelial dystrophy and other corneal dystrophies (e.g., including lattice, granular, macular, and map-dot fingerprint), pseudophakic and/or aphakic bullous keratopathy, ocular cicatricial pemphigoid, or any combination thereof. In some embodiments, the disease is conjunctivitis, blepharitis, a cataract, or any combination thereof.

In some embodiments, the disease is an ocular posterior segment disease. Non-limiting examples of ocular posterior segment diseases include glaucoma, eye stroke (e.g., retinal artery occlusions & retinal vein occlusions), age-related macular degeneration, macular edema, retinal detachment, ocular hypertension, or any combination thereof.

### Methods of Preparing

Provided herein are methods of preparing any of the compositions disclosed herein. The methods include dissolving GelMA and HAGM in a first solution including the visible light-activated photoinitiator; and mixing a second solution including the micelles with the dissolved GelMA and HAGM in the first solution.

In some embodiments, the micelles are formulated separately from the precursor hydrogel composition (e.g., the first solution). In some embodiments, the methods include mixing a therapeutic agent with the one or more micelles (e.g., in the second solution) prior to forming the first solution or separately from the first solution. In some embodiments, the micelles are formulated to encapsulate a therapeutic agent. For example, and as described in Example 1, in some embodiments, drug loaded-micelles are formed by self-assembly via a solvent evaporation method. In some embodiments, the methods include dissolving the copolymers to be used in the synthesis of micelles with an organic solvent (e.g., acetone). Next, the methods can include adding the therapeutic agent to the copolymer solution and vigorously mixing (e.g., vortexing) until fully mixed. Next, methods can include adding the polymer/therapeutic agent solution to a buffer (e.g., ammonium acetate buffer) while stirring. In some embodiments, the method further includes stirring the mixture and then heating the mixture. In some examples, the mixture is stirred at room temperature (e.g., at about 20° C. to about 25° C.) for a period of time (e.g., about 30 minutes). In some embodiments, the mixture is heated to a temperature of about 45° C. (e.g., about 40° C. to about 50° C.). In some embodiments, after a period of time (e.g., 2 hours), the mixture is slowly cooled down to room temperature and stirred overnight. In some embodiments, the methods include centrifuging the micelle solution to remove any unencapsulated therapeutic agent.

In some embodiments, the methods include preparing the precursor hydrogel composition (e.g., the first solution) by mixing GelMA and HAGM with a photoinitiator (PI) solution. In some embodiments, the PI solution is prepared by dissolving one or more photoinitiators (e.g., in powder form) in a buffer (e.g., phosphate buffered saline (PBS)). In some embodiments, the methods include thoroughly mixing GelMA and HAGM in the PI solution and incubating at an elevated temperature (e.g., at about 45° C. to about 55° C.) for a period of time (e.g., about 12 hours or overnight).

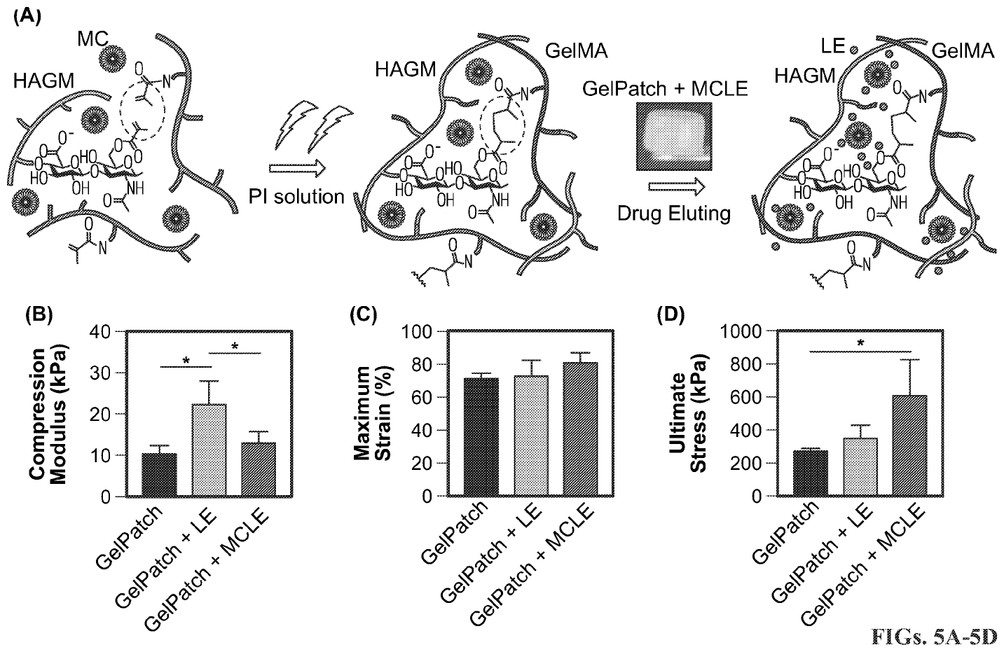

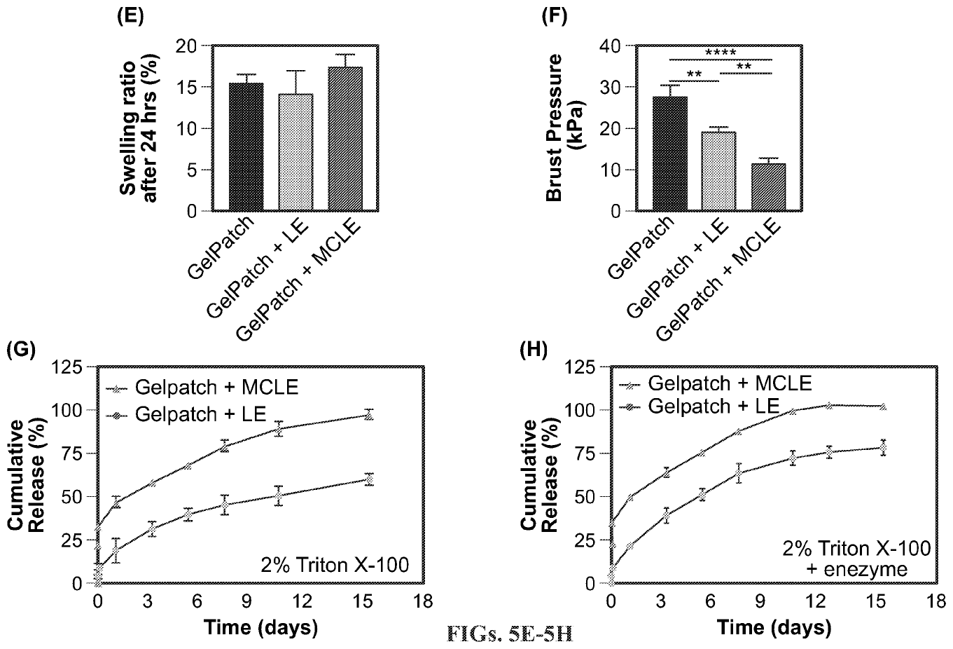

In some embodiments, after complete dissolution of the precursor hydrogel composition components, and as shown in FIG. 5A, the methods include mixing the synthesized micelle solution (e.g., the second solution) with the precursor hydrogel composition (e.g., the first solution). In some embodiments, the first solution and the second solution further include a buffer, a solvent, water, or any combination thereof. In some embodiments, the methods include photo-crosslinking the micelle and precursor hydrogel composition solutions (e.g., the second and first solutions, respectively) by exposing the composition to a visible light. In some embodiments, the composition (e.g., the first and second solutions) changes from a solution form to a hydrogel form after photo-crosslinking.

### EXAMPLES

Certain embodiments of the present disclosure are further described in the following examples, which do not limit the scope of any embodiments described in the claims.

### Example 1—Synthesis of Micelles

### Materials

4,4-azobis(4-cyanopentanoic acid) (ABCPA), poly(ethylene glycol) methyl ether (Mw 5000 g/mol) (mPEG), N,N′-Dicyclohexylcarbodiimide (DCC), 4-dimethylaminopyridine (DMAP), and p-toluenesulfonic acid, L-lactide, N-(2-hydroxypropyl)methacrylamide (HPMAm), Tin(II) 2-ethylhexanoate (SnOct₂), and 4-methoxyphenol were purchased from Sigma-Aldrich. All solvents: tetrahydrofuran (THF), dichloromethane (DCM), dimethylformamide (DMF), acetonitrile (ACN), and acetone were provided by Sigma-Aldrich or Fisher Chemical. Nuclear magnetic resonance (NMR) solvents: chloroform-d (CDCl₃), deuterated dimethyl sulfoxide (DMSO-d6) and deuterium oxide (D₂O) were purchased from Cambridge Isotope Laboratories, Inc. Gelatin from porcine skin (Gel strength 300, type A), methacrylic anhydride, hyaluronic acid sodium salt from Streptococcus equi, glycidyl methacrylate (GM), Eosin Y disodium salt, triethanolamine (TEA) and N-vinylcaprolactam (VC), Triton X-100 were all purchased from Sigma-Aldrich.

Synthesis of Macroinitiator mPEG₂-ABCPA

Macroinitiators mPEG₂-ABCPA were synthesized through an esterification of mPEG and ABCPA using DCC as a coupling reagent and 4-(dimethylamino)pyridinium 4-toluenesulfonate (DPTS), which was made by 1:1 molar ratio of DMAP and p-toluenesulfonic acid in TIF) as a catalyst. In brief, 1 equiv of ABCPA (0.280 g), 2 equiv of PEG (10 g), and 0.3 equiv of DPTS (36.7 mg of DMAP and 57.3 mg of p-toluensulfonic acid each separately dissolved in 1 mL of TIF) were dissolved in 50 mL of dry DCM with stirring on ice bath. Vacuum and nitrogen alternating cycles were repeated three times. Next, 3 equiv of DCC (0.619 g) were dissolved in 50 mL of dry DCM and dropwise added to PEG solution under nitrogen atmosphere. After the addition of DCC, the ice bath was removed allowing the mixture to react at room temperature. After 16 h, the reaction mixture was filtered to remove 1,3-dicyclohexyl urea salts and was dried under vacuum to remove solvents. Then, the remaining product was re-dissolved in water, stirred for 2 h, and dialyzed against water for 72 h at 4° C. The final white product was obtained by freeze-drying and was analyzed by gel permeation chromatography (GPC) in DMF and proton nuclear magnetic resonance (H NMR) spectroscopy in CDCl₃.

### Synthesis of Monomer HPMAm-Lacn

A mixture of 1 equiv of L-lactide (1 g), 1 equiv of HPMAm (0.993 g), 0.01 equiv of SnOct₂ (28.1 mg, 1 mol % relative to HPMAm), and 0.001 equiv of 4-methoxyphenol (0.86 mg, 0.1 mol % relative to HPMAm) were added in a round bottom flask. Vacuum and nitrogen alternating cycles were repeated three times to remove air. Then, the mixture was heated to 130° C. with stirring for 1 h and allowed to cool to room temperature.

The purification was done through a silica column chromatography. The reaction mixture was first dissolved in small amount of ethyl acetate (EtOAc) and dry-loaded on a silica column. 90% EtOAc/Hexane solvent system was used to run the column entirely. Thin Layer Chromatography (TLC) was used to analyze the separation. The fractions containing HPMAm-Lacn were collected, and after solvent evaporation, the identity of obtained fractions was established by ¹H-NMR in CDCl₃. ¹H NMR for monomer HPMAm-Lacₙ(CDCl₃, 400 MHz): Chemical shift (δ, ppm)=6.32-6.04 (b, 1H, H₃), 5.71 (s, 1H, H₁), 5.35 (s, 1H, H_{1′}), 5.2-5.0 (m, H₅, H₈), 4.36 (q, 1H, H₉), 3.62 (m, 1H, H₄), 3.37 (m, 1H, H_{4′}), 1.96 (s, 3H, H₂), 1.50 (d, 6H, H₇, H₁₀), 1.27 (d, 3H, H₆).

Synthesis of Copolymer mPEG-b-p(HPMAm-Lacn)

The mPEG-b-p(HPMAm-Lacn) was synthesized by radical polymerization using mPEG₂-ABCPA as macroinitiator and HPMAm-Lacₙ as monomer. Monomer to macroinitiator feed ratio was 150:1. A mixture of HPMAm-Lacn and mPEG₂-ABCPA were dissolved in dry ACN. The concentration of macroinitiator plus monomer was 300 mg/mL in ACN. The resulting solution was degassed by freeze-pump-thaw method and then heated to 70° C. with stirring for 24 h. After 24 h, the reaction was cooled to room temperature and diluted in a small amount of ACN (˜2 mL). The product in the solution was precipitated by dropwise addition to an excess of cold diethyl ether (˜45 mL) in a 50 mL vial. After centrifugation at 3000 rpm for 15 min, white pellet was obtained. Diethyl ether wash followed by centrifugation was repeated three times. Followed by dissolution in water, product solution was dialyzed (MWCO 12-14 kDa) against water and finally recovered by freeze drying. The final product was analyzed by GPC and ¹H-NMR. ¹H NMR for copolymer mPEG-b-p(HPMAm-Lacₙ) (CDCl₃, 400 MHz): δ=6.6 (b, H₁), 5.3-4.8 (b, H₃, H₄), 4.39 (b, H₅), 3.64 (b, PEG CH₂—CH₂), 3.3-2.7 (b, H₂), 2.4-0.4 (the rest of the protons).

### Preparation of Unloaded and Drug Loaded Micelles

Drug loaded MCs were formed by self-assembly via a solvent evaporation method. Briefly, 10 mg of copolymers mPEG-b-p(HPMAm-Lacn) were dissolved in 1 mL of acetone. Various amounts of drugs (0.25, 0.5, 1 and 2 mg) were then added to the copolymer solution and vortexed until fully mixed. Next, polymer/drug cocktail solution was quickly added to 1 mL of ammonium acetate buffer (120 mM, pH 5) with stirring. The mixture was stirred at room temperature for 30 min and then heated to 45° C. After 2 h, the mixture was slowly cooled down to room temperature and stirred overnight. The next day, the MC solution was centrifuged at 4,000 rpm at 22° C. for 10 min to remove unencapsulated drugs. LE, PA, and DEX were three drugs of interest. 30 mg/mL of drug stock solutions were prepared in DMSO. Unloaded MCs were prepared with the same procedure without the addition of drugs.

### Example 2—Fabrication of GelPatch

### Synthesis of GelMA

GelMA was synthesized as described previously. In brief, 10% (w/v) gelatin from porcine skin (Bloom 300, type A, Sigma) was dissolved in Dulbecco's phosphate buffered saline (DPBS) and 8% (v/v) methacrylic anhydride was added dropwise at 55° C. The mixture was allowed to react for 3.5 h under continuous stirring. The reaction was stopped by two times dilution in DPBS and was dialyzed against water at 50° C. for 5 days. Finally, the resulting solution was frozen at −80° C. for 24 h and freeze-dried for 5 days to yield GelMA.

### Synthesis of HAGM

Hyaluronic acid (HA) was modified with GM to form HAGM using the following protocol. In brief, 1% (w/v) of HA (1.6 MDa, Sigma) was dissolved in 200 mL deionized water for 12 h under continuous stirring. After it fully dissolved, 8 mL triethylamine, 8 mL GM, and 4 g of tetrabutyl ammonium bromide (TBAB) were added in order separately and thoroughly mixed for 1 h before the next addition. Following complete dissolution, the reaction was allowed to continue overnight (16 h, 22° C.) and was finally completed by incubation at 60° C. for 1 h. After cooling to room temperature, the solution was then precipitated in 20 times excess volume of acetone (4 L) as white solid fibers. The precipitate was then dissolved in water, dialyzed for 2 days and lyophilized.

### Preparation of Unloaded and Loaded GelPatch

GelPatch prepolymer was prepared by mixing GelMA and HAGM with a photoinitiator (PI) solution. This light-sensitive PI solution was prepared by dissolving 0.5 mM Eosin Y disodium salt, 1.86% (w/v) TEA and 1.25% (w/v) VC in phosphate buffered saline (PBS). 1 N hydrochloric acid was used to adjust the pH of PI solution to 8. Then, 7% (w/v) GelMA and 3% (w/v) HAGM were thoroughly mixed in PI solution and incubated at 50° C. overnight. After complete dissolution, the final GelPatch prepolymer solutions were crosslinked for 4 min with visible light (450 to 550 nm) by using an LS1000 Focal Seal Xenon Light Source (100 mW/cm², Genzyme). GelPatch containing free LE (GelPatch+LE) and GelPatch containing LE loaded MCs (GelPatch+MCLE) were prepared with an additional step of physically mixing LE powder or MC solutions with dissolved GelMA and HAGM in PI solution before crosslinking.

### Example 3—Characterization of GelPatch and Micelle Compositions

### ¹H NMR Spectroscopy and Formulas

### ¹H NMR Analysis of Macroinitiator, Monomer and Copolymer

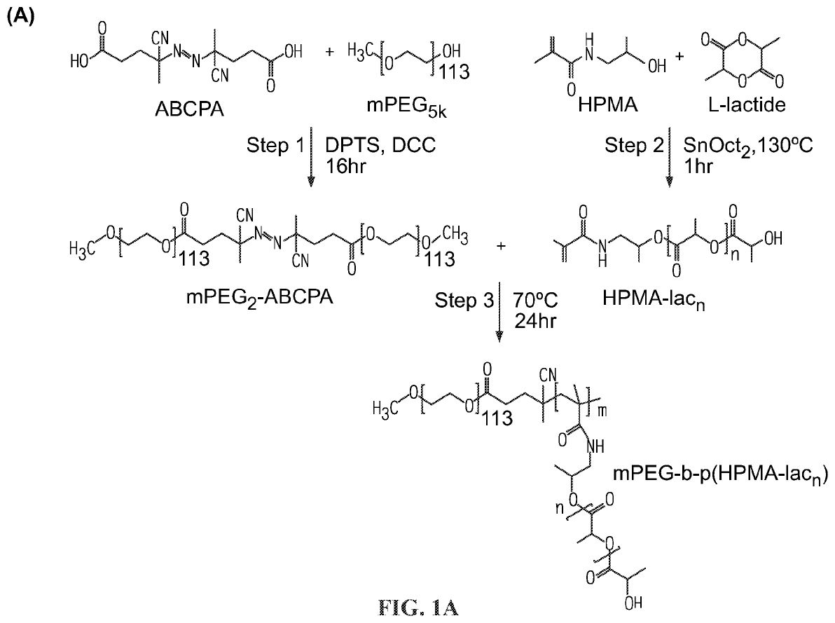

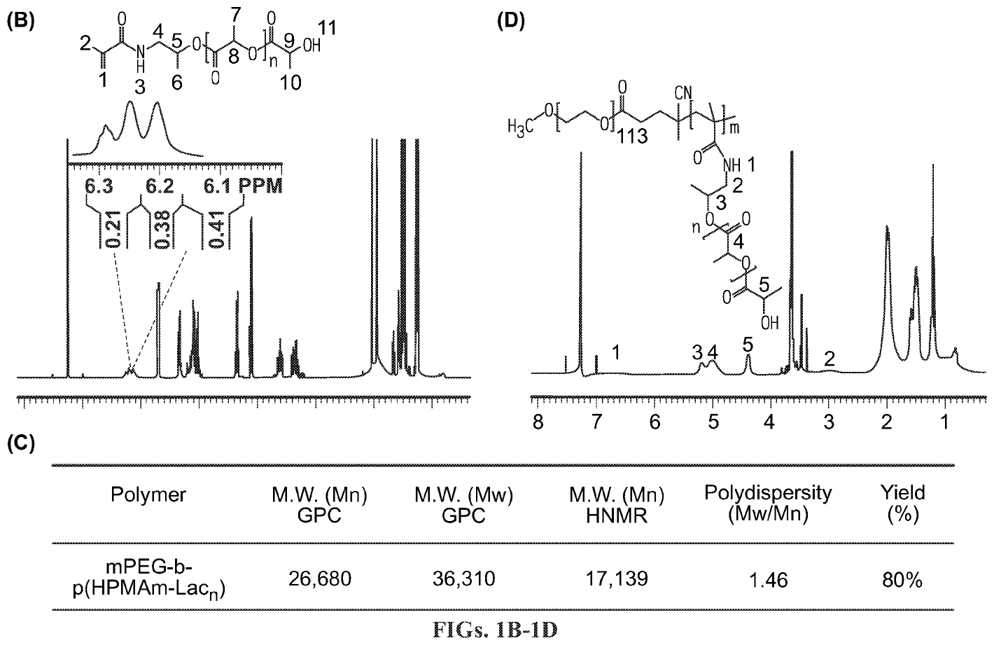

The ¹H NMR spectra of macroinitiator, monomer, and copolymer were obtained in CDCl₃ using a Brucker AV 400 MHz NMR Spectrometer (2 sec delay and 64 scans). The chemical shift of CDCl₃ at 7.26 ppm was used as reference line. The average molecular weight of HPMAm-Lacₙ(Mw_{ave_HPMAm-Lac}_{ₙ}), the number of HPMAm-Lacₙ repeating units (m) and the average molecular weight of mPEG-b-p(HPMAm-Lacₙ) (Mn) were determined by ¹H NMR, using the following equations:

$$( 1 ) {Mw}_{ave\_HPMAm - {Lac}_{n}} = \% {Lac}_{2} \times 287.31 + \% {Lac}_{3} \times 359.38 + \% {Lac}_{4} \times 431.44 \qquad ( Eq . 1 )$$

$$( 2 ) m = \frac{{I}_{C O - C H ( C {H}_{3} ) - O H}}{{I}_{P E {G}_{5 k}} / 454} \qquad ( Eq . 2 )$$

$$( 3 ) Mn = {Mw}_{P E {G}_{5 k}} + m \times M {w}_{ave\_HPMAm - {Lac}_{n}} \qquad ( Eq . 3 )$$

Where I_{CO-CH(CH}_{₃}_{)-OH} is the value of the integration of the methine proton next to the hydroxyl group (FIG. 1D, H₅, δ=4.39 ppm). I_{PEG}_{₅ₖ}/454 is the ratio of the integration of PEG₅ₖ proton to the average number of protons per PEG₅ₖ chain.

### ¹H NMR Analysis of Ge/MA and HAGM

The gelatin and GelMA were dissolved in DMSO-d6 and HAGM was dissolved in D₂O at a concentration of 10 mg/mL and at a temperature of 50° C. The ¹H NMR spectra were recorded with a Brucker AV 400 MHz NMR Spectrometer (10 sec delay and 64 scans). The degree of methacrylation (DM) of GelMA was defined as the ratio of methacrylate groups to the free amine groups in gelatin prior to the reaction. The vinyl protons on methacrylamide grafts gave rise to two peaks at δ=5.62 and 5.29 ppm. The peak areas of methylene protons of lysine groups (δ=2.75 ppm) in the spectra of gelatin and GelMA were integrated separately. The DM of GelMA was calculated from the following equation.

$$DM ( \% ) = 1 - \frac{{I}_{lysine} ( GelMA )}{{I}_{lysine} ( Gelatin )} \times 100 \% \qquad ( Eq . 4 )$$

The DM of HAGM was defined as the amount of methacryloyl groups per one HA disaccharide repeating unit. The two vinyl protons on methacrylate groups had chemical shifts of 6.16 and 5.16 ppm. The DM was calculated from the ratio of the relative peak integrations of the methyl protons of methacrylate groups (δ=1.93 ppm) to the methyl protons of amide groups (δ=2 ppm) on HA.

$$DM ( \% ) = \frac{{I}_{{H}_{3}} ( methyl Hs on GM ) / 3}{{I}_{{H}_{4}} ( methyl Hs on HA ) / 3} \times 100 \% \qquad ( Eq . 5 )$$

### Gel Permeation Chromatography (GPC)

Analysis of the mPEG₂-ABCPA macroinitiator and mPEG-b-p(HPMAm-Lacn) copolymer was performed using a Waters System (Waters Associates Inc., Milford, MA) with refractive index (RI) using two serial PLgel 5 μm MIXED-D columns (Polymer Laboratories) and THF as eluent. The flow rate was 0.7 mL/min (45 min run time) and the temperature was 25° C. The molecular weights of the synthesized polymers were determined by GPC analysis using RI detector and standards to calculate the number average molecular weight (Mn), the weight average molecular weight (Mw), and polydispersity index (PDI; (Mw/Mn)).

### Dynamic Light Scattering (DLS)

Freshly prepared micellar dispersions were diluted 25 times with 10 mM HEPES, pH 7.0 (final concentration 400 g/mL) and their sizes were analyzed with a Malvern Zetasizer Nano dynamic light scattering. Standard operating procedure parameters: 10 runs, 10 sec/run, three measurements, no delay between measurements, 25° C. with 120 sec equilibration time. Collection parameters: S26 lower limit=0.6, upper limit=1000, resolution=high, number of size classes=70, lower size limit=0.4, upper size limit=1000, lower threshold=0.05, upper threshold=0.01. Data is representative of three replicate measurements.

### Zeta Potential

Zeta potential of the MCs was determined using a Malvern Zetasizer Nano-Z (Malvern Instruments, Malvern, UK) with universal ZEN 1002 ‘dip’ cells and DTS (Nano) software (version 4.20) at 25° C. Zeta potential measurements were performed in 10 mM HEPES at pH 7.4 at a final polymer concentration of 400 μg/mL.

### Transmission Electron Microscopy (TEM)

The sample preparation for cryo-TEM was performed in a temperature and humidity-controlled chamber using a fully automated vitrification robot (FEICo., Hillsboro, OR). A thin aqueous film of MC solution was formed on a Quantifoil R 2/2 grid (Quantifoil Micro Tools GmbH, Jena, Germany) at 22° C. and at 100% relative humidity. This thin film was rapidly vitrified by shooting the grid into liquid ethane. The grids with the vitrified thin films were transferred into the microscope chamber using a Gatan 626 cryo-transfer/cryo-holder system (Gatan, Inc., Pleasanton, CA). Micrographs were taken using a CM-12 transmission microscope (Philips, Eindhoven, The Netherlands) operating at 120 kV, with the specimen at −170° C. and using low-dose imaging conditions.

### Determination of Encapsulation Efficiency and Loading Capacity

The amount of loaded drugs (LE, PA and DEX) in the polymeric MCs was determined by using High Performance Liquid Chromatography (HPLC). Taking LE as an example, after centrifugation of the MC solution, the unencapsulated LE pellet was redissolved in 1 mL of ACN. The concentration of this solution was measured by HPLC using a 70-90% ACN/water gradient solvent system at 242 nm (60-80% gradient at 243 nm for PA and 50-80% gradient at 239 nm for DEX). LE dissolved in ACN (concentration from 0.1 mg/ml to 1 mg/ml) was used for calibration. The encapsulation efficiency (EE) and loading capacity (LC) were calculated as follows:

$$EE \% = 1 - \frac{a mount of unloaded drugs}{a mount of drugs used for loading} \times 100 \% \qquad ( Eq . 6 )$$

$$LC \% = 1 - \frac{a mount of unloaded drugs}{a mount of copolymer used for loading} \times 100 \% \qquad ( Eq . 7 )$$

### Mechanical Characterization

For the unconfined compression test, 75 μL of hydrogel precursor solution was pipetted into a polydimethylsiloxane (PDMS) cylindrical mold (diameter: 6 mm; height: 2.5 mm). The resulting solution was photocrosslinked via exposure to visible light for 4 min. After photocrosslinking, the dimensions of the hydrogels were measured using a digital caliper. The compression tests were conducted using an Instron 5542 mechanical tester. The crosslinked hydrogel cylinders were placed between the compression plates and compressed at a rate of 1 mm/min until failure. The slope of the stress-strain curves was obtained and reported as the compression modulus (N=3).

### In Vitro Burst Pressure Test

Burst pressure resistance (e.g., adhesion strength) of the composite hydrogel formulations was measured by using the ASTM F2392-04 standard according to the following method. Briefly, collagen sheet made out of porcine intestine (4×4 cm) was placed in between two stainless steel annuli from a custom-built burst pressure device, which consists of a metallic base holder, pressure meter, syringe pressure setup, and data collector. A hole (2 mm diameter) was created through the sheet and was sealed (photocrosslinked) by applying 30 μL of hydrogel precursor solution. Next, the airflow was applied into the system, and the maximum burst pressure was recorded until detachment from the collagen sheet or hydrogel rupture. The burst pressure resistant was measured using a pressure sensor connected to a computer. Three replicates were performed for each hydrogel sample.

### Measurement of Swelling Ratio

Hydrogel samples were prepared as described in previous section. The weight of each hydrogel sample (N=3) was measured following photocrosslinking and after 24 h in DPBS at 37° C. The swelling ratio was then calculated according to the equation below, where W₀ is the weight of the sample just after photocrosslinking and W₁ is the final weight of the sample after 24 h incubation.

$$Swelling ratio ( \% ) = \frac{{W}_{1} - {W}_{0}}{{W}_{1}} \times 100 \qquad ( Eq . 8 )$$

In Vitro Release Profile from Micelles and from GelPatch

The release profiles of LE, PA and DEX from the polymeric MCs were examined by a dialysis method. Taking LE as an example, 1 mL of LE loaded MC solution was pipetted into a dialysis bag (MWCO 12-14 kDa). The releasing medium was prepared with a solution of 2% Triton X-100 in DPBS. The dialysis bag was immersed in 10 mL of the releasing medium with stirring at 300 rpm at 37° C. Samples (5 mL) of the receiving medium were drawn periodically and 5 mL of fresh releasing medium were added back to keep the volume constant. The concentration of LE in the different samples was measured using HPLC method mentioned in previous section. Calibration was done using LE (concentration from 0.005 mg/mL to 0.1 mg/mL) in 2% Triton X-100 in DPBS.

In vitro release profiles of LE from GelPatch+MCLE and GelPatch+LE were measured using the same releasing medium. A 250 μL of GelPatch+MCLE precursor solution was pipetted into cylindrical mold and photocrosslinked for 4 min. The gel cylinder was then immersed in 10 mL of releasing medium (2% Triton X-100 in DPBS) in an incubator shaker at 75 rpm at 37° C. Samples (5 mL) of the receiving medium were drawn periodically and fresh releasing medium were added back to keep the volume constant. The concentration of LE in the different samples (N=3) was measured by HPLC. Additionally, in vitro release profile in the presence of enzymes were studied by adding 5 μg/mL of collagenase and 5 μg/mL of hyaluronidase on top of 2% Triton X-100 releasing medium with all other procedures remaining the same.

### In Vitro Biocompatibility of GelPatch and MC Loaded GelPatch (GelPatch+MC)

hTCEpi cells were cultured at 37° C. and 5% CO₂ in KBM™ basal media (00192151) supplemented with KGM-Gold™ Keratinocyte SingleQuots™ Kit (00192152). The cells were seeded on the surface of the hydrogel scaffolds. Briefly, 10 μL of GelPatch precursor solutions were spread and photocrosslinked on a 3-(trimethoxysilyl) propyl methacrylate (TMSPMA)-coated glass slide, providing 1×1 cm² surface areas of hydrogels. Samples (N≥3) were placed in 24 well-plate and hTCEpi cells were seeded on the hydrogel surface (10′ cells per sample). After incubation of the seeded samples in a humid incubator with 5% CO₂ for 20 min at 37° C., 400 μL of media was added to each well and incubated. The media was replaced with fresh media every other day.

The viability of cultured cells on the gel scaffolds at day 1 and day 3 was evaluated using a Live/Dead™ Viability/Cytotoxicity Kit (Invitrogen) as stated by the manufacturer's instructions. Briefly, a solution of calcein AM at 0.5 L/mL (green color, viable cells) and ethidium homodimer at 2 L/mL (red color, non-viable cells) in DPBS was used to stain the cells. After 15 min of incubation, samples were washed with DPBS, and cells were imaged using a fluorescence optical microscope (Primovert, Zeiss). The collected images were analyzed using ImageJ software to quantify the cell viability (%) by dividing the number of live cells by the total number of live and dead cells.

Proliferation and metabolic activity of cells were determined using a PrestoBlue assay (Invitrogen) at day 1, 3, and 7 after culture according to the manufacturer's instructions. Briefly, seeded samples were incubated with a media solution containing 10% PrestoBlue reagent for 45 min with 5% CO₂ at 37° C. The Fluorescence intensity of the solution was determined using a plate reader (BioTek) at 540 nm (excitation)/600 nm (emission).

The morphology of the cells and their expansion were assessed through staining of F-actin filaments with Alexa Fluor 594-phalloidin (Invitrogen) to visualize the cytoskeleton and cell nuclei with DAPI. Briefly, cells were fixed by incubating with 4% (w/v) paraformaldehyde for 15 min, then permeabilized using 0.3% (v/v) Triton in DPBS for 10 min and blocked with 1% (w/v) bovine serum albumin (BSA) in DPBS for 30 min at room temperature. Samples were serially incubated with phalloidin (1:400 dilution in 0.1% BSA) and DAPI (1:1000 dilution) solution for 45 min and 1 min, respectively. The samples were washed and imaged using the Zeiss fluorescent microscope.

### In Vivo Studies on GelPatch and GelPatch+MC

### Subcutaneous Implantation in Rats

All the in vivo studies were approved by the ICAUC (protocol 2018-076-01C) at University of California Los Angeles (UCLA). Male Wistar rats (200-250 gr) were purchased from Charles River Laboratories (Boston, MA, USA). Anesthesia was achieved by inhalation of isoflurane (2-2.5%), followed by subcutaneous meloxicam administration (5 mg/kg). After anesthesia, eight one-cm incisions were made on the dorsal skin of rats, and small subcutaneous pockets were made using a blunt scissor. GelPatch+MC, as well as pristine GelPatch as a control were formed using a cylindrical compression mold and then were lyophilized. The lyophilized hydrogels were sterilized under UV light for 10 min. The sterile hydrogels were then implanted into the subcutaneous pockets, and incisions were closed with 4-0 polypropylene sutures (AD Surgical). At day 7 and 28 post-implantation, the rats were euthanized, and the hydrogels were explanted with the surrounding tissues for histological assessment.

Histological analyses were performed on the explanted hydrogels to investigate the inflammatory responses caused by the implanted hydrogels. After explantation of the samples with the surrounding tissues, they were fixed in 4% (v/v) paraformaldehyde for 4 h and incubated in 15% and 30% sucrose, respectively (at 4° C., overnight). Samples were then embedded in Optimal Cutting Temperature compound (OCT), frozen in liquid nitrogen, and sectioned by using Leica CM1950 cryostat machine. Sections (8 μm thickness) were mounted on positively charged glass slides using DPX mountant (Sigma) for Hematoxylin and Eosin (H&E) staining and Masson's Trichrome (MT) staining, and ProLong™ Gold antifade reagent (Thermo fisher scientific) for immunofluorescence (IF) staining. The slides were then processed for H&E and MT staining (Sigma) according to manufacturer instructions. IF staining was also performed on mounted samples as previously reported. Anti-CD68 (ab125212) (Abcam) was used as primary antibody, and Goat-anti Rabbit IgG (H+L) secondary antibody conjugated to Alexa Fluor® 594 (Invitrogen) was used as a detection reagent. All samples were then stained using 4′,6-diamidino-2-phenylindole (DAPI) and the imaging was performed using ZEISS Axio Observer Z1 inverted microscope.

### Statistical Analysis

Results were presented as means±SD (*P<0.05, **P<0.01, ***P<0.001 and ****P<0.0001). One-way or two-way analysis of variance (ANOVA) t test was performed followed by Tukey's test for statistical analysis (GraphPad Prism 8.0).

### Example 4—Synthesis and Characterization of Copolymer

Diblock copolymer mPEG-b-p(HPMAm-Lacn) composed of a hydrophobic HPMAm-Lacₙ block and a hydrophilic mPEG block was synthesized by radical polymerization with macroinitiator mPEG₂-ABCPA in a high yield of 80% (FIG. 1A). The monomer HPMAm-Lacₙ was synthesized by ring-opening oligomerization of L-lactide using SnOct₂ as a catalyst. The monomer mixtures were purified through silica column chromatography to remove residual HPMAm and obtain a mixture of HPMAm-Lac₂ to HPMAm-Lac₄ in a form of light-yellow colored viscous solution. From ¹H NMR spectrum, the percentage of HPMAm-Lac₂, HPMAm-Lac₃ and HPMAm-Lac₄ were calculated to be 41%, 38% and 21%, respectively, based on the integration ratio of amide protons (—NH—) in 6.1˜6.3 ppm region (FIG. 1B). In addition, the PDI (Mw/Mn) of copolymer was measured to be 1.46 based on GPC analysis (FIG. 1C), which was within the expected range for the polymers synthesized via free radical polymerization. The average number of repeating units of HPMAm-Lacn were calculated to be ˜32 (Eq. 2) and therefore, by adding the molecular weight of mPEG block, the average molecular weight of the synthesized block copolymer was estimated to be ˜17,139 Da from ¹H NMR spectrum (FIG. 1D). The synthesized mPEG-b-p(HPMAm-Lacn) block copolymers were then used as building blocks to form MCs, which solubilize our selected anti-inflammatory drugs.

### Example 5—Micelle (MC) Formation and Core Interaction with Corticosteroids

mPEG-b-p(HPMAm-Lacₙ) MCs can be synthesized and used to solubilize several hydrophobic therapeutics, such as paclitaxel, vitamin K and an MRI contrast agent. mPEG-b-p(HPMAm-Lacn) MCs have tunable biodegradability due to the hydrolysis of the lactate side chains under physiological conditions, which can enable sustained release of loaded therapeutics via diffusion. In addition, the PEG shell of MCs offered several advantages including drug protection, prolonged systemic circulation, and reduced macrophage uptake. Their small size can allow better tissue penetration via enhanced permeability and retention (EPR) effect and facilitated overcoming physiological barriers. The MCs may also be applied in cancer therapy. Herein, for the first time, mPEG-b-p(HPMAm-Lacn) MCs were utilized to solubilize hydrophobic anti-inflammatory drugs for the treatment of ocular injuries.

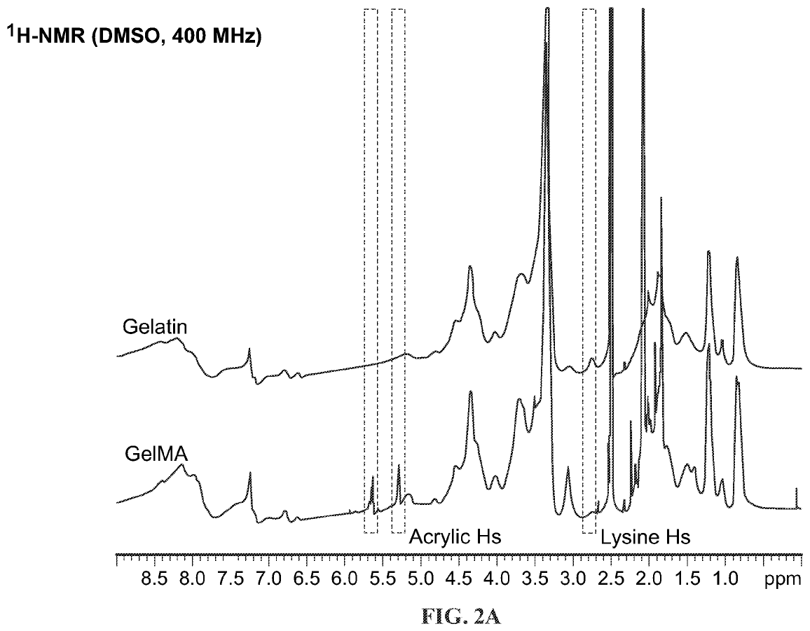

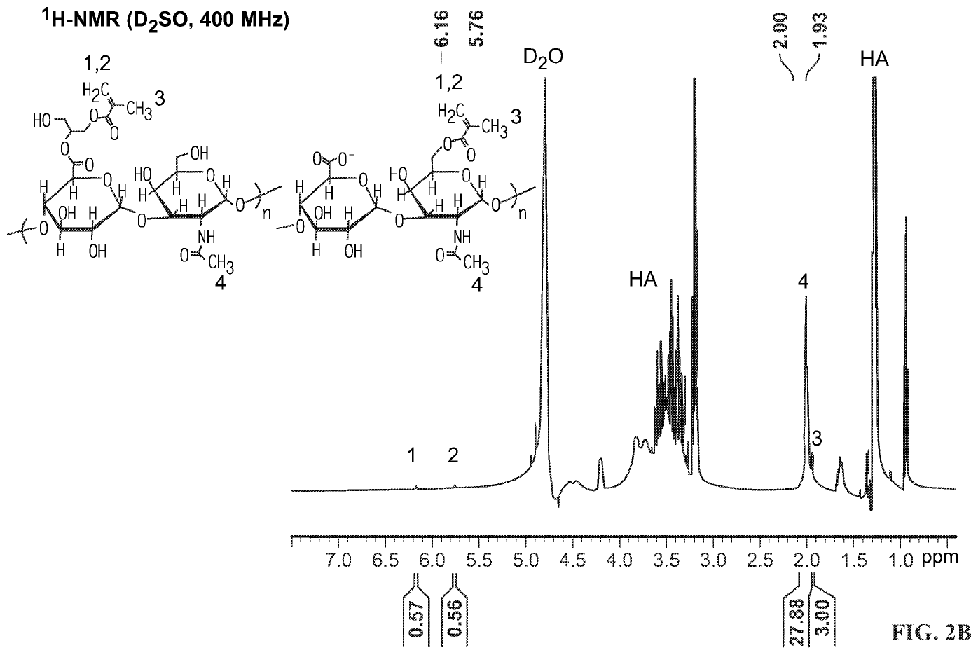

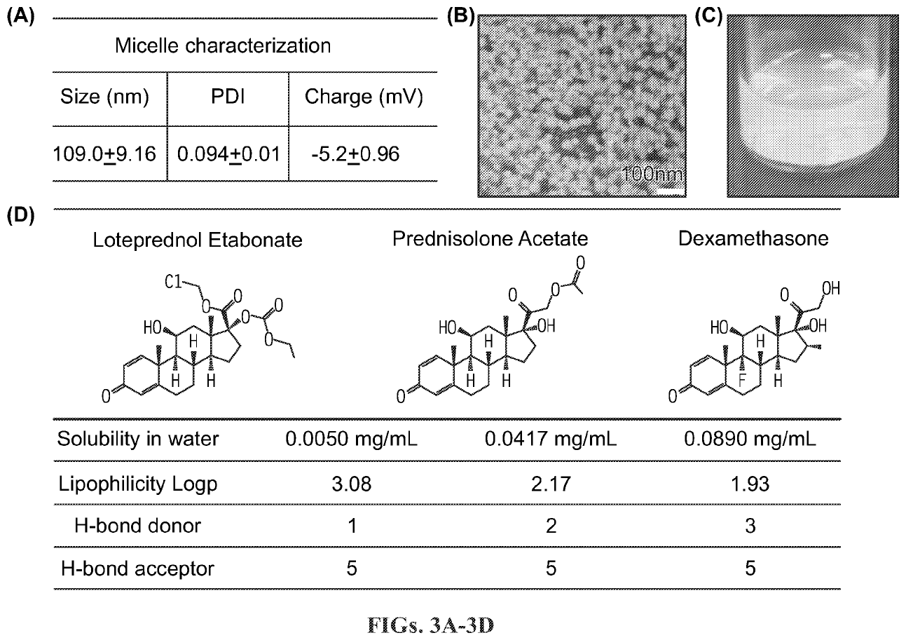

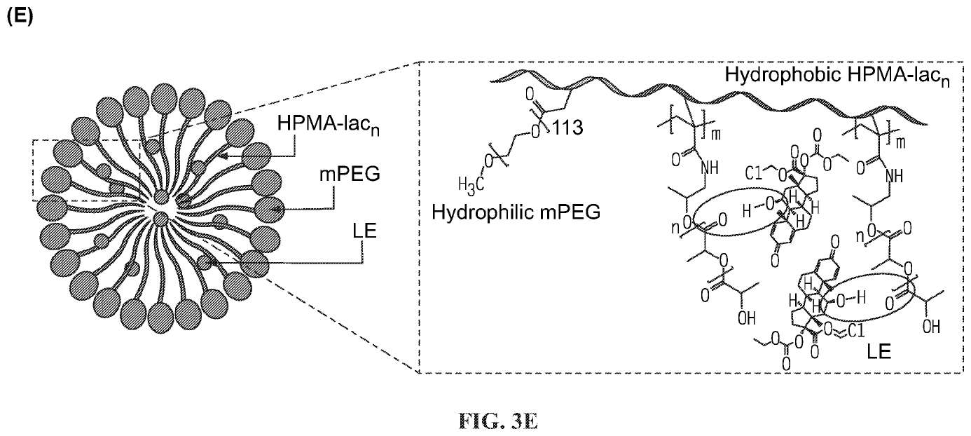

MCs were formed by self-assembly via a solvent evaporation method. mPEG-b-p(HPMAm-Lacn) copolymers were firstly dissolved in acetone and were fast addition to aqueous solution composed of ammonium acetate buffer solution. During the evaporation of acetone, amphiphilic copolymers formed core-shell structures with hydrophobic HPMAm-Lacₙ block clustering away from aqueous phase and hydrophilic mPEG orienting towards aqueous phase. The average size of unloaded MCs was 109.0±9.16 nm with a PDI of 0.094±0.01 measured by DLS (FIG. 3A). The surface charge density of unloaded MCs was determined to be −5.2±0.96 mV via Zetasizer. TEM image of unloaded MCs was shown in FIG. 2B. The dispersion of MCs of mPEG-b-p(HPMAm-Laca) was opalescent and homogeneous (FIG. 3C) [43].

The table in FIG. 3D summarized the physicochemical properties and molecular structure of three types of corticosteroids that were formulated within the MCs. Loteprednol etabonate (LE), Prednisolone acetate (PA) and Dexamethasone (DEX) are FDA-approved anti-inflammatory compounds to treat ocular anterior segment diseases. They all have similar core structures with different functional groups attached to the cyclopentane ring in addition to a fluoride at the carbon 9 (C-9) position for DEX and a hydrogen at C-9 position for LE and PA. All of three are lipophilic molecules (Log P>0) and have poor solubility in water (<0.1 mg/mL). Among these three compounds, LE has only one H-bond donor and the highest Log P (Log P=3.08), which explains its lowest solubility in water (0.0005 mg/mL).

Drug loaded MCs were formed by an additional step of mixing drugs with the synthesized copolymers in acetone before encounter with the aqueous phase. During the process of acetone evaporation, free floating hydrophobic drugs were slowly clustered with the hydrophobic blocks of copolymers to form drug loaded MCs. Drug loading was achieved via both H-bonding and hydrophobic interactions between the drugs and copolymers functional groups (FIG. 3E). The 2-h heating process accelerated molecule movement, which increased the likelihood of drugs interacting with hydrophobic blocks and getting loaded into the hydrophobic core of MCs.

### Example 6—Characterization of Drug Loaded MCs

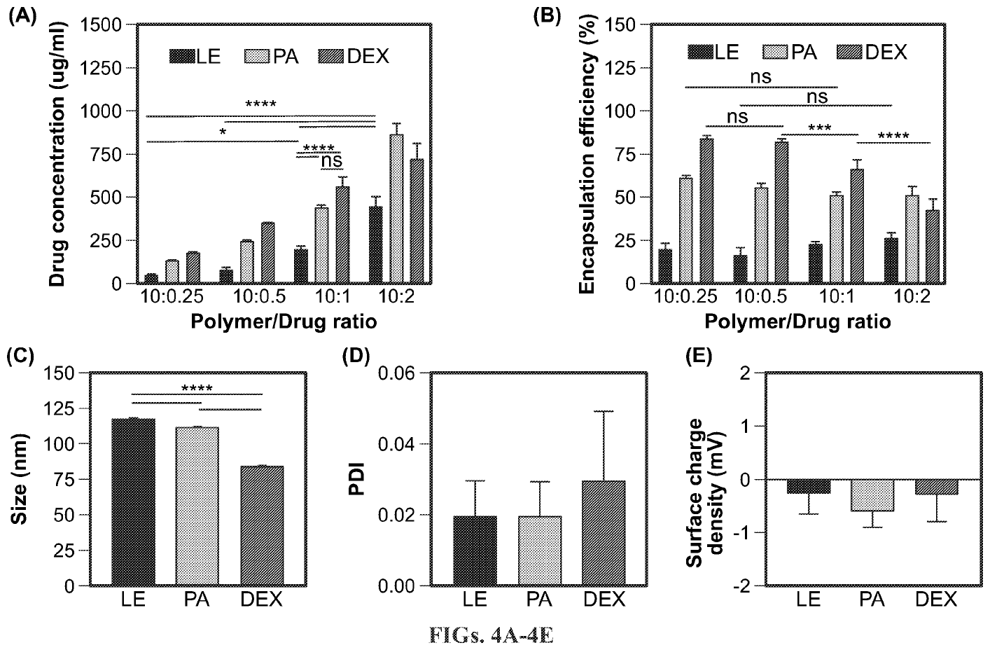

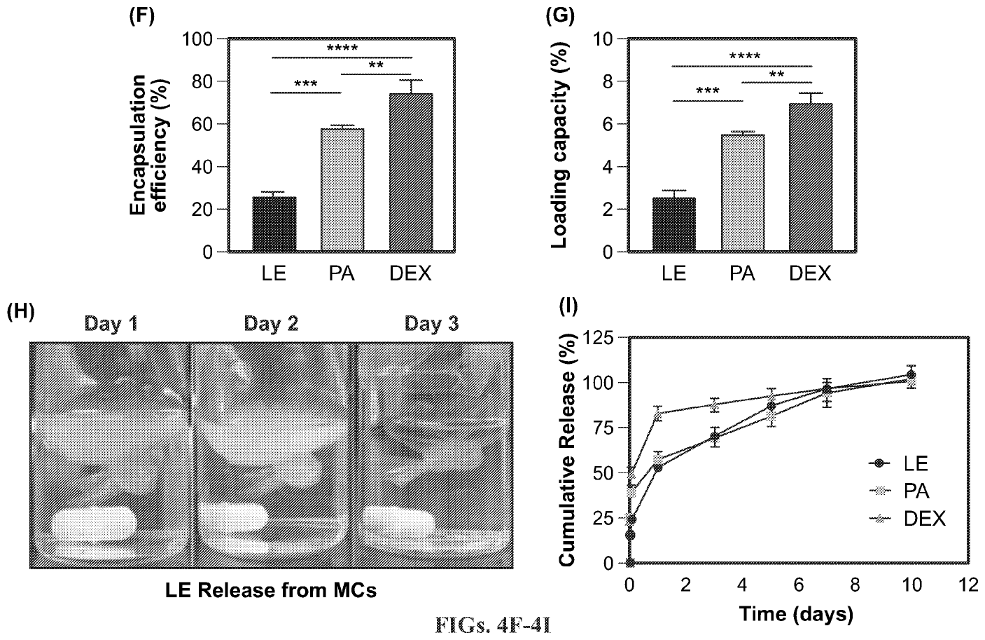

The amount of drug loaded inside the micellar formulation varied with applying different polymer/drug ratios (w/w). For all drug candidates (LE, PA, and DEX), various amounts composed of 0.25, 0.5, 1 and 2 (mg/mL), were applied with a fixed concentration (10 mg/mL) of polymers to form drug loaded MCs. The final concentration of drug loaded in 1 mL of MC solution was measured using High Performance Liquid Chromatography (HPLC), and the results were shown in FIG. 4A. With an increase in initially applied drug concentration in the formulation from 0.25 mg to 2 mg, the final concentration of drug loaded inside MCs increased from 55.7±10.0 μg/mL to 589.2±75.5 μg/mL for LE, from 171.7±4.2 μg/mL to 1154.0±108.6 μg/mL for PA, and from 235.9±5.0 μg/mL to 956.7±150.3 μg/mL for DEX. No significant difference in encapsulation efficiency (EE) % of LE and PA with various polymer/drug ratios was detected, while EE % for DEX decreased from 92.5±1.7% to 47.8±7.5% with increase in applied concentration of DEX from 0.5 mg/mL to 2 mg/mL, respectively (FIG. 4B). Based on the results of loaded drug concentrations (μg/mL) and EE %, 10:1 polymer/drug ratio was selected for further characterization steps including size, PDI, and surface charge of drug loaded MCs.

The hydrodynamic size of drug loaded MCs were measured by dynamic light scattering (DLS) analysis. The size of the LE, PA and DEX MCs was measured to be 117.30±0.30 nm, 111.40±0.94 nm and 84.30±0.34 nm, respectively (FIG. 4C). An increase in size of LE and PA drug-loaded MCs was observed compared to the unloaded MCs (109.0±9.16 nm). In addition, a correlation between decrease in size of drug loaded MCs with increase in number of H-bond donor groups on the drug molecules was observed. DEX loaded MCs had the smallest size among the three due to the most H-bonding interactions (H-bond donors=3) between the hydroxyl groups on DEX and the ester oxygen of the hydrophobic MC core, which explained the decreased size of DEX loaded MCs compared to the unloaded MCs. PDI values for three drug-loaded MCs were 0.02±0.01 (LE), 0.02±0.01 (PA) and 0.03±0.02 (DEX), which indicated desired homogeneity and monodisperse of the MC formulations (FIG. 4D). The drug loaded MCs had net neutral surface charge density of −0.24±0.40 mV for LE, −0.59±0.31 for PA, −0.27±0.51 for DEX (FIG. 3E), which was desired for a slow degradation rate to serve as sustained drug release carriers. Regarding encapsulation efficiency (EE) and drug loading capacity (LC), LE showed EE % and LC % of 25.5±2.8% and 2.5±0.3%, respectively at 10:1 polymer/drug ratio (FIGS. 3F and 3G). PA and DEX loaded MCs showed higher values of EE % 57.8±2.1% and 74.6±6.0%, respectively and higher values of LC % 5.5±0.2% and 6.9±0.5%, respectively. These results were in alignment with the hypothesis that in addition to hydrophobic interactions at the core of the MCs, higher number of H-bond donors on the drug molecules supported more H-bonding interactions with hydrophobic core of MCs, which consequently led to higher EE %, LC % and smaller MC size.

### Example 7—In Vitro Release of Drugs from MCs

One important aspect of this study was to achieve a sustained release of drugs to ocular surfaces in order to reduce the eyedrop instillation frequency and improve patient compliance. To this end, in vitro release of LE, PA and DEX from MCs was assessed via a dialysis method under sink conditions. Since LE, PA and DEX had extremely low aqueous solubility, it was difficult to maintain the molecular dispersion using Dulbecco's phosphate buffered saline (DPBS) alone. A large amount of release media was required to keep drugs solubilized, but the drug concentration might be too low to be detectable by HPLC. Therefore, 2% Triton X-100 was added as surfactant in DPBS buffer to better solubilize the released drug molecules. The addition of non-ionic surfactants may not induce MC destabilization and may not form mixed MCs due to the different chemical properties of surfactants and copolymers. The appearance change of LE loaded MCs submerged in the release media was observed over 5 days (FIG. 4H). The visible light scattering properties of MCs altered over time as the size and integrity of MCs in the dialysis bag changed. This was likely due to the hydrolysis of the lactate chains of copolymers, which led to hydrophilization and the swelling of the core of the MCs. It was found that LE, PA and DEX loaded mPEG-b-p(HPMAm-Lacn) MCs released their full contents (e.g., drug payload) over the time period of 10 days (FIG. 4I). An initial burst release was observed for all three formulations which indicates the presence of residual free drug molecules in the formulation. DEX loaded MCs had the fastest initial release, where 50.0% of DEX was released after 2 h and 83.1% of DEX was released after 24 h. PA loaded MCs showed a slower release rate as compared to 39.4% of PA release after 2 h and 57.3% after 24 h. LE loaded MCs showed the slowest release profile, where 24.2% of LE was released after 2 h and 53.1% after 24 h. The desired administration of topical corticosteroids is an initial burst release combined with gradual and slow release over time. Among all the release profiles, LE loaded MCs showed the desired release profile for ocular drug delivery compared to other drug loaded MCs. In addition, from pharmacology point of view, LE stands out from PA and DEX molecules because it features an ester at the carbon 20 (C-20) position instead of a ketone. The C-20 ester allows LE to be metabolized into inactive metabolites after exerting therapeutic effects, thereby avoiding adverse effects associated with intraocular pressure (IOP) relative to ketone-based corticosteroids. Therefore, LE encapsulated MCs were chosen to be incorporated into the ocular adhesive hydrogel patch and further investigated its properties.

### Example 8—Fabrication and Characterization of Unloaded and MCs Loaded GelPatch

Our ocular drug delivery platform, GelPatch, was a composite adhesive hydrogel composed of gelatin methacryloyl (GelMA) and hyaluronic acid-glycidyl methacrylate (HAGM) which was loaded with the LE loaded MCs. ¹H NMR analysis was performed to determine the degree of methacrylation (DM) of GelMA and HAGM. Comparing ¹H NMR spectra of gelatin and GelMA, new peaks at δ=5.62 and 5.29 ppm were corresponding to the two protons of methacrylate double bond (FIG. 2A). In addition, the decreased integration of lysine peaks at δ=2.75 ppm further confirmed the reaction of gelatin with methacrylic anhydride. The DM of GelMA was calculated to be 61% based on the percentage of consumption of lysine peaks (Eq. 4). HA was reacted with glycidyl methacrylate to form HAGM. The DM of HAGM was defined as the amount of methacrylate groups per one HA disaccharide repeating unit. The DM of HAGM was calculated to be 11% based on the ratio of the relative peak integration of methacrylate methyl protons (δ=1.93 ppm) to HA's methyl protons (δ=2.0 ppm) (Eq. 5, FIG. 2B). The synthesized GelMA (7%, w/v) and HAGM (3%, w/v) were then mixed with the photoinitiator (PI) solution, which consisted of Eosin Y initiator, triethanolamine (TEA) and N-vinylcaprolactam (VC). The precursor solution of GelPatch was a viscous liquid. Finally, GelPatch hydrogel was formed by photocrosslinking the mixture under visible light for 4 min. To prepare GelPatch+MCLE, LE loaded MCs were mixed with dissolved GelMA and HAGM in PI solution before photocrosslinking as illustrated in FIG. 5A. Crosslinked hydrogel cylinders were obtained and used to evaluate the mechanical properties and in vitro swelling ratio.

The mechanical properties of GelPatch hydrogels were determined through compression tests (FIGS. 5B, 5C, 5D). There was no significant difference in the maximum strain among GelPatch, GelPatch containing free LE (GelPatch+LE) and GelPatch containing LE loaded MCs (GelPatch+MCLE) (FIG. 5C). However, the loading of free LE doubled the compression modulus from 10.30±2.03 kPa to 22.39±5.52 kPa compared to GelPatch, while the loading of LE loaded MCs did not significantly change the compression modulus which was measured as 13.02±2.67 kPa. The increased compression modulus of GelPatch+LE indicated its increased stiffness of the crosslinked hydrogel. This can be caused by the presence of aggregated LE particles dispersed as crystalline domains inside the GelPatch hydrogel due to its poor aqueous solubility, whereas for GelPatch+MCLE, there was no solubility issue, since MCs well solubilized LE before loading into GelPatch. Furthermore, the ultimate stress of GelPatch increased from 276.0±15.52 kPa to 610.8±215.43 kPa after the addition of LE loaded MCs and did not change after the addition of free LE. These results indicated that the dispersion of MCs within the hydrogel matrix can strengthen the resistance to the deformation of GelPatch hydrogels.

In addition to the mechanical properties, the swelling ratio was also evaluated. GelPatch itself had a swelling ratio of 15.38±1.06% in DPBS at 37° C. after 24 h. It was found that the addition of free LE and LE loaded MCs had no significant effect on the swelling ratio of GelPatch, and the values were measured to be 14.01±20.81% and 17.32±1.52% for GelPatch+LE and GelPatch+MCLE, respectively, as shown in FIG. 5E.

The adhesive properties of hydrogel were essential, since the hydrogel would be applied as drug delivery matrix loaded with solubilized drug molecules that can adhere to the ocular surface and directly deliver anti-inflammatory drugs to the site of inflammation. Therefore, the adhesive properties of drug loaded/unloaded GelPatch to the biologic surfaces were evaluated. In vitro burst pressure tests were performed based on a modified ASTM standard test (F2392-04) for GelPatch, GelPatch+LE and GelPatch+MCLE. The results showed that the burst pressure of GelPatch decreased from 27.7±2.6 kPa to 19.35±0.95 kPa after the addition of free LE and decreased to 11.6±1.1 kPa after the addition of LE loaded MCs, as shown in FIG. 5F. The adhesion to collagen sheet, which was used as a biological substrate in this test, was due to the various chemical bond formation and physical interactions at the substrate/hydrogel interface. Although the addition of LE loaded MCs changed the intermolecular interactions within GelPatch as well as surface layer composition, which reduced the interface adhesion, GelPatch+MCLE still maintained an improved adhesive strength compared to several commercially available surgical sealants such as Evicel®, CoSeal™, DuraSeal®, and fibrin sealants.

### Example 9—In Vitro Release of LE from GelPatch

In vitro release profiles of LE from GelPatch+LE and GelPatch+MCLE were obtained in 2% Triton X-100 in DPBS with and without the presence of enzymes. FIG. 5G showed that 88.9% of LE was released from GelPatch+MCLE in 10 days and 100% was released after 15 days. On the other hand, only 50.2% of LE was released from GelPatch+LE in 10 days and 59.3% was released after 15 days. The slower release of LE from GelPatch+LE was due to the fact that LE remained in crystalline form inside GelPatch and its release was entirely based on the swelling and degradation of GelPatch by creating opportunities for Triton X-100 surfactant to solubilize LE into the releasing media. However, for the GelPatch+MCLE, the release of LE can be described as two continuous processes. LE loaded MCs were able to diffuse out slowly from GelPatch and then the hydrolysis of MCs happened in the release media where LE then got released. Simultaneously, the entrapped MCs could be hydrolyzed even slower within GelPatch and later LE would diffuse out from GelPatch directly. These two processes explained the slower LE release profile from GelPatch+MCLE compared to the LE release profile just from MCs. In order to assess the release profile in the presence of enzymes to mimic the real eye environment, we added 5 μg/mL of collagenase and 5 μg/mL of hyaluronidase in the 2% Triton X-100 release media. FIG. 5H showed that the overall release of LE in the presence of enzymes was faster from both GelPatch+LE and GelPatch+MCLE. 99.5% of LE was released from GelPatch+MCLE in 10 days and full release within 12 days; For GelPatch+LE, 72.2% of LE was released in 10 days and 78.5% after 15 days. The increased release rate in the presence of enzyme was due to the faster enzymatic degradation of GelPatch, which resulted in decreasing polymer network density and increasing pore size. Therefore, MCs diffused out faster from GelPatch leading to a faster release of LE observed in FIG. 5H. The release profile data supported the study objective of the sustained release of anti-inflammatory LE to treat ocular injuries.

### Example 10—In Vitro Biocompatibility of the GelPatch Scaffolds

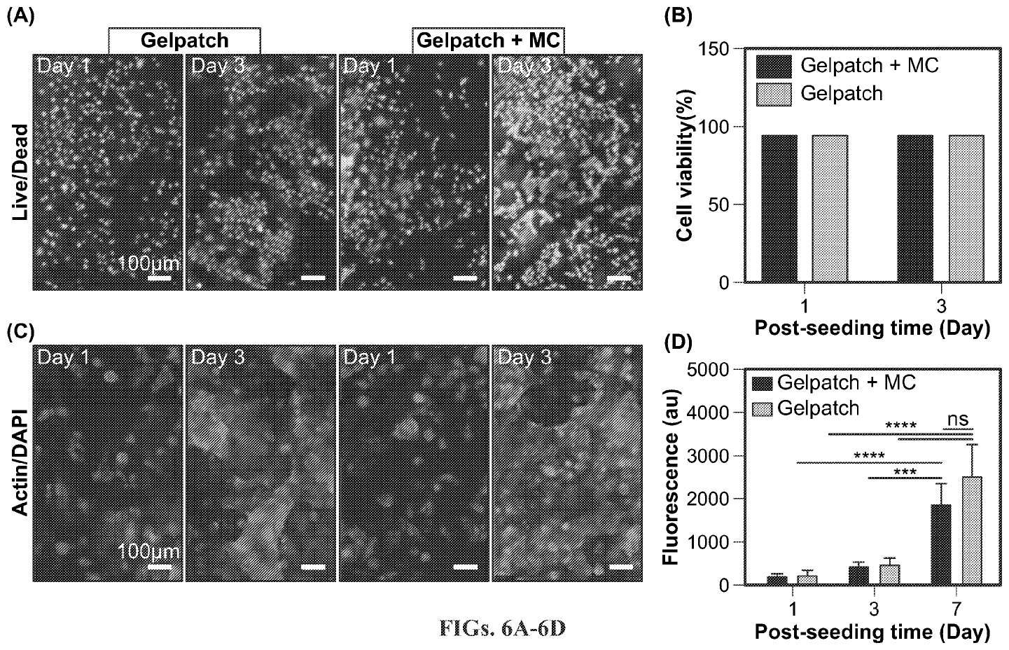

To evaluate the biocompatibility of GelPatch and GelPatch+MC (without LE), the viability and metabolic activity of the seeded cells on the crosslinked gel samples were investigated through Live/Dead assay and PrestoBlue assay (at day 1, 3, and 7). The micrographs of stained cells by Live/Dead assay at day 1 and day 3 showed high viability of cells (>90%) seeded on both GelPatch+MC and GelPatch samples at the early stage of their culture (FIGS. 6A and 6B). In addition, the morphology of the cultured cells on the hydrogels was evaluated using fluorescent staining F-actin in the cytoskeleton of cells on day 1 and day 3. The assembly of F-actin cytoskeleton of cells in fluorescent micrographs showed that the cells spread, adhered, and proliferated on the surfaces of both GelPatch+MC and GelPatch samples, indicating the in vitro biocompatibility of the samples for cell adherence and growth (FIG. 6C). The metabolic activity of cultured hTCEpi cells on samples through PrestoBlue assay showed a consistent increase over 7 days without a significant difference to the control GelPatch group, which confirmed the biocompatibility of GelPatch+MC (FIG. 6D).

### Example 11—In Vivo Biocompatibility and Biodegradation of Gelpatch and GelPatch+MC Using a Rat Subcutaneous Model

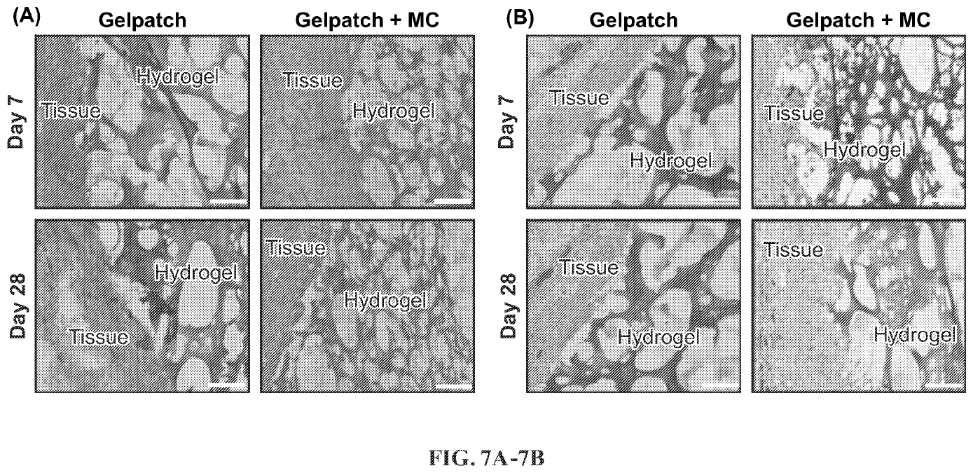

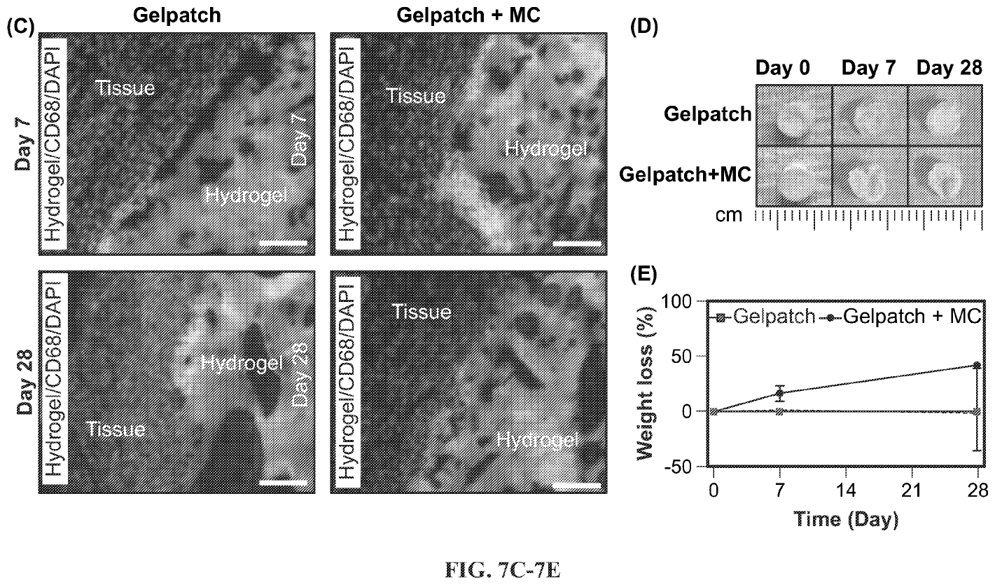

Lastly, subcutaneous implantations of GelPatch and GelPatch+MC in rats were performed to investigate their in vivo biocompatibility and biodegradation. The Hematoxylin and Eosin (H&E) staining images showed a small amount of cell infiltration in both GelPatch and GelPatch+MC (FIG. 7A). Based on Masson's Trichrome (MT) staining in FIG. 7B, no significant fibrosis was detected in both hydrogels. In addition, immunofluorescence analysis of subcutaneously implanted hydrogels demonstrated the presence of macrophages (CD68) at day 7, but they significantly reduced at day 28 (FIG. 7C). For in vivo biodegradation, our results suggested that there was no statistically significant change after 28 days, as demonstrated by visual inspection (FIG. 7D) and measurements in the weight loss of the samples (FIG. 7E). The large error bar seen in the percentage of weight loss of GelPatch samples (FIG. 7E) might be due to the variance of entrapment of adjacent tissues in the hydrogel samples, as the presence of red color shown in the images of the lyophilized hydrogels post-implantation (FIG. 7D). These results suggested that GelPatch encapsulated with MCs was biocompatible and was able to retain in a good shape after 28 days, which would allow sustained full release of drugs before GelPatch degraded.

### Example 12—GelPatch Entrapping LE Loaded MCs for Treatment of Ocular Injuries

Anti-inflammatory drug eluting adhesive patches were developed for treatment of ocular injuries. As drug carriers, mPEG-b-p(HPMAm-Lacn) MCs successfully solubilized hydrophobic corticosteroids through H-bonding and hydrophobic interactions and provided sustained release of these drugs as demonstrated in the in vitro release studies. In addition, photocrosslinkable composite adhesive GelPatch showed appropriate mechanical strength, adhesion and swelling properties as ocular drug delivery platform. The incorporation of LE loaded MCs in GelPatch had no significant effect on the mechanical properties or the swelling properties of the composite hydrogel. Despite a small decrease in burst pressure, GelPatch+MCLE still maintained an improved adhesive strength compared to several commercially available surgical sealants and was able to achieve a sustained full release of LE in 15 days without enzymes and in 12 days in the presence of collagenase and hyaluronidase. Moreover, in vitro cell studies showed that MC loaded GelPatch had good biocompatibility and supported cellular adhesion, proliferation, and growth. In vivo studies further proved the in vivo biocompatibility and biodegradation of the engineered drug eluting adhesives to ensure the full release of anti-inflammatory drugs over 28 days. The developed non-invasive, adhesive, and biocompatible GelPatch containing LE loaded MCs may provide several advantages over conventional drug delivery methods such as, but not limited to, improved patient compliance, site-targeted delivery, and lower dosage requirements, as well as circumventing the drawbacks of microneedles and current available adhesives. The MC loaded GelPatch system had great capacity to incorporate other hydrophobic therapeutics and could become a promising ocular drug delivery platform for treatment of different ocular anterior segment diseases and injuries.

### OTHER EMBODIMENTS

It is to be understood that while certain embodiments have been described within the detailed description, the present disclosure is intended to illustrate and not limit the scope of any embodiment defined by the scope of the appended claims. Other aspects, advantages, and modifications are within the scope of the appended claims.

What is claimed is:

1. A composition, comprising:
one or more micelles encapsulating a hydrophobic therapeutic agent;
gelatin methacryloyl (GelMA);
hyaluronic acid-glycidyl methacrylate (HAGM); and
a visible light-activated photoinitiator.

2. The composition of claim 1, wherein the one or more micelles are block copolymer micelles comprising a hydrophobic core and a hydrophilic shell.

3. The composition of claim 2, wherein the block copolymer is poly(ethylene glycol)-b-poly(N-(2-hydroxypropyl) methacrylamide-oligolactate) (mPEG-b-p(HPMAm-Lacₙ)).

4. The composition of any one of claims 1-3, wherein the hydrophobic therapeutic agent is a first therapeutic agent, and the composition further comprises a second therapeutic agent.

5. The composition of claim 4, wherein the first and/or second therapeutic agents are anti-inflammatory therapeutic agents.

6. The composition of claim 5, wherein the first and/or second therapeutic agents are loteprednol etabonate (LE), prednisolone acetate (PA), dexamethasone (DEX), or any combination thereof.

7. The composition of any one of claims 1-6, wherein the one or more micelles have a diameter ranging from about 50 nanometers (nm) to about 150 nm.

8. The composition of any one of claims 1-7, wherein the GelMA is present at a concentration ranging from 3% (w/v) to about 14% (w/v).

9. The composition of any one of claims 1-8, wherein the HAGM is present at a concentration ranging from about 0.5% (w/v) to about 3% (w/v).

10. The composition of claims 8 or 9, wherein the composition has a sustained therapeutic release profile over a period of about 15 days at most.

11. The composition of any one of claims 1-10, wherein the photoinitiator comprises Eosin Y, triethanolamine (TEA), N-vinylcaprolactam (VC), or any combination thereof.

12. The composition of any one of claims 1-11, wherein the composition is in a form of a solution or a hydrogel.

13. The composition of any one of claims 1-12, wherein the composition further comprises a pharmaceutically acceptable carrier or excipient.

14. The composition of any one of claims 1-13, wherein the composition is formulated for topical use.

15. The composition of any one of claims 1-14, wherein the one or more micelles are present at a concentration of about 5 mg/ml to about 30 mg/ml.

16. A method of treating an ocular disease or an ocular injury in an eye of a subject, the method comprising:
contacting the eye of the subject with the composition of any one of claims 1-15; and
photo-crosslinking the composition by exposing the composition to a visible light.

17. The method of claim 16, wherein the ocular disease is an ocular anterior segment disease or an ocular posterior segment disease.

18. The method of claim 17, wherein the ocular disease is conjunctivitis, blepharitis, glaucoma, or a cataract.

19. The method of claim 18, wherein the ocular injury is an ocular surface injury or an injury or trauma caused by an ocular surgery.

20. The method of any one of claims 16-19, wherein the visible light has a wavelength of about 400 nanometers (nm) to 800 nm.

21. A method of preparing the composition of any one of claims 1-15, the method comprising:
dissolving the GelMA and the HAGM in a first solution comprising the visible light-activated photoinitiator; and
mixing a second solution comprising the one or more micelles with the dissolved GelMA and HAGM in the first solution.

22. The method of claim 21, further comprising mixing a therapeutic agent with the one or more micelles prior to forming the first solution.

23. The method of any one of claims 21 to 22, wherein the first solution and the second solution further comprise a buffer, a solvent, water, or any combination thereof.

24. The method of any one of claims 21 to 23, further comprising incubating the first solution in the presence of the GelMA and the HAGM for at least about 12 hours prior to mixing with the second solution.

25. The method of any one of claims 21 to 24, further comprising photo-crosslinking the first and second solutions after mixing the second solution with the dissolved GelMA and HAGM in the first solution by exposing the composition to a visible light.

26. The method of claim 25, wherein the composition changes from a solution form to a hydrogel form after photo-crosslinking the first and second solutions.
